(19) United States
(12) Patent Application Publication
(10) Pub. No.: US 2023/0000969 A1
(43) Pub. Date: Jan. 5, 2023

(54) IMPROVED VACCINES FOR RECURRENT RESPIRATORY PAPILLOMATOSIS AND METHODS FOR USING THE SAME

(71) Applicant: Inovio Pharmaceuticals, Inc., Plymouth Meeting, PA (US)

(72) Inventors: David B. Weiner, Merion, PA (US); Jian Yan, Wallingford, PA (US)

(21) Appl. No.: 17/771,736

(22) PCT Filed: Oct. 26, 2020
(86) PCT No.: PCT/US20/57314

§ 371 Date: Apr. 25, 2022

Related U.S. Application Data
(60) Provisional application No. 62/925,283, filed on Oct. 24, 2019.

(51) Int. Cl. A61K 39/12; A61P 31/20

(52) U.S. Cl. CPC A61K 39/12; A61P 31/20; A61K 2039/53

(57) ABSTRACT

The use of anti-HPV immunogens and nucleic acid molecules that encode them for the treatment and prevention of RRP are disclosed. Pharmaceutical composition, recombinant vaccines comprising DNA plasmid and live attenuated vaccines are disclosed as well methods of inducing an immune response to treat or prevent RRP are disclosed.

### CROSS-REFERENCE TO RELATED APPLICATIONS

This application claims priority to and the benefit of U.S. Provisional Application No. 62/925,283 filed Oct. 24, 2019, which is incorporated herein by reference in its entirety.

### FIELD OF THE INVENTION

The present invention relates to improved human papillomavirus (HPV) vaccines, improved methods for inducing immune responses, and for prophylactically and/or therapeutically immunizing individuals against recurrent respiratory papillomatosis (RRP).

### BACKGROUND OF THE INVENTION

Human Papilloma Virus associated (HPV+) malignancies are an emerging global epidemic (Gradishar et al., Journal of the National Comprehensive Cancer Network: JNCCN. 2014; 12(4):542-90). HPV associated aerodigestive precancerous lesions and malignancies may occur in the oropharynx, larynx, and upper respiratory tract. While the role of HPV6 in the etiology of a majority of aerodigestive malignancies remains unclear, its role is widely accepted as being causally implicated in recurrent respiratory papillomatosis (RRP) (Mounts et al., Proc Natl Acad Sci USA. 1982; 79(17):5425-9; Gissmann et al., Proc Natl Acad Sci USA. 1983; 80(2):560-3; Bonagura et al., APMIS. 2010; 118(6-7):455-70), the most common benign tumor of the laryngeal epithelium. RRP is rare, with an incidence rate estimated at 1.8 per 100,000 adults in the United States (Winton et al., The New England journal of medicine. 2005; 352(25):2589-97). Although most lesions are benign, some undergo malignant transformation, and patients with RRP have a higher risk of developing laryngeal neoplasias and carcinomas (Omland et al., PloS one. 2014; 9(6):e99114).

The clinical course of RRP can vary widely amongst affected individuals. Selection of treatment, including active monitoring without treatment, surgery, radiation therapy, or a combination, depends on a number of factors. Usually, repeated surgical removal of papillomas for symptomatic management remains the mainstay of treatment (Derkay et al., Otolaryngol Clin North Am. 2019; 52(4):669-79). In a few cases, malignant transformation may occur, which is usually associated with a dismal prognosis. A few such patients with malignant disease may be candidates for salvage therapies, including potentially definitive surgery (Richey et al., Otolaryngology—head and neck surgery: official journal of American Academy of Otolaryngology-Head and Neck Surgery. 2007; 136(1):98-103). Selected patients in this setting may benefit from radiation although the morbidity of this approach is substantial (Mendenhall et al., American journal of clinical oncology. 2008; 31(4):393-8). Recently, a Phase II study of pembrolizumab in patients with RRP demonstrated a response rate of 43%, and supports a rationale for immunotherapeutic management of RRP (Pai et al., Journal of Clinical Oncology. 2019; 37(15_suppl):2502).

Thus, there is a need in the art for improved compositions and methods for treatment or prevention of RRP. The present invention satisfies this unmet need.

### SUMMARY OF THE INVENTION

Aspects of the invention provide compositions comprising at least one nucleotide sequence comprising an HPV6 E6-E7 fusion antigen, and uses thereof for the treatment or prevention of RRP.

Another aspect provides compositions comprising one or more nucleotide sequences encoding an HPV6 E6-E7 fusion antigen selected from the group consisting of: nucleotide sequence that encodes SEQ ID NO:2; a nucleotide sequence that is at least 95% homologous to a nucleotide sequence that encodes SEQ ID NO:2; a nucleotide sequence that is at least 95% homologous to a fragment of a nucleotide sequence that encodes SEQ ID NO:2. In some embodiments, the nucleotide sequences encoding the HPV6 E6-E7 fusion antigen are without a leader sequence at 5′ end that is a nucleotide sequence that encodes SEQ ID NO:4.

In another aspect of the invention, there are provided compositions comprising one or more nucleotide sequences encoding an HPV6 E6-E7 fusion antigen selected from the group consisting of: SEQ ID NO:1; a nucleotide sequence that is at least 95% homologous to SEQ ID NO:1; a fragment of SEQ ID NO:1; a nucleotide sequence that is at least 95% homologous to a fragment of SEQ ID NO:1. In some embodiments, the nucleotide sequences encoding the HPV6 E6-E7 fusion antigen are without a leader sequence at 5′ end that has nucleotide sequence SEQ ID NO:3.

The nucleotide sequences provided can be a plasmid.

In additional aspects, provided are pharmaceutical compositions comprising the disclosed nucleotide sequences.

In some aspects, there are methods of treating or preventing RRP in an individual by inducing an effective immune response in an individual, comprising administering to said individual a composition comprising one or more of the nucleotides sequences provided. The methods preferably include a step of introducing the provided nucleotide sequences into the individual by electroporation.

In some aspects, the method further comprises administering to the individual a composition comprising an adjuvant. In one embodiment, the method further comprises administering to the individual a composition comprising a nucleic acid molecule encoding IL-12. For example, in certain embodiments, the method further comprises administering to the individual a composition comprising a nucleic acid molecule encoding one or more of: p35 and p40 subunits of IL-12.

In some aspects, the method comprises administering to the individual a nucleic acid molecule comprising a nucleotide sequence encoding one or more of: p35 and p40 subunits of IL-12. In one embodiment, nucleotide sequence encoding p35 comprises a nucleotide sequence selected from the group consisting of: a nucleotide sequence that encodes SEQ ID NO:6; a nucleotide sequence that is at least 95% homologous to a nucleotide sequence that encodes SEQ ID NO:6; a fragment of a nucleotide sequence that encodes SEQ ID NO:6; and a nucleotide sequence that is at least 95% homologous to a fragment of a nucleotide sequence that encodes SEQ ID NO:6. In one embodiment, the nucleotide sequence encoding p40 comprises a nucleotide sequence selected from the group consisting of: a nucleotide sequence that encodes SEQ ID NO:8; a nucleotide sequence that is at least 95% homologous to a nucleotide sequence that encodes SEQ ID NO:8; a fragment of a nucleotide sequence that encodes SEQ ID NO:8; and a nucleotide sequence that is at least 95% homologous to a fragment of a nucleotide sequence that encodes SEQ ID NO: 8.

### BRIEF DESCRIPTION OF THE FIGURE

FIG. 1: Comparative 3D models of the HPV6 E6 and HPV6 E7 SynCon antigens. E6 is modeled as a monomer and the ordered C-terminal region of E7 is modeled as a homodimer. The disordered N-terminal is indicated in the figure. Both are visualized in ribbon format with side chains and a transparent solvent-accessible surface. Zinc finger motifs on both models are annotated.

FIG. 2: Interferon gamma is produced by HPV6 E6 and HPV6 E7 specific T cells in RRP patients. Subjects 603 (upper panel) and 604 (lower panel) were tracked for the ability to produce Interferon gamma in an ELISpot assay longitudinally across the study. E6 specific activity is displayed in blue dashed lines, E7 specific activity is displayed in a solid blue line, the sum of both antigens is displayed in a solid black line. Long Term Follow Up (LTFU) timepoints are noted and described in relative to time following the completion of Dose 4.

FIG. 3: INO-3106 Activates HPV6-specific cytotoxic lymphocytes cells in RRP patients. Flow cytometry was performed to assess activation marker expression on HPV6-specific CD8+ T cells taken from subjects before and after immunotherapy. Expression of CD137 and CD38 before (upper panel) and after (lower panel) treatment with INO-3106 in patient 604 are noted in the left column. Expression of Ki67 and CD69 before (upper panel) and after (lower panel) treatment with INO-3106 in patient 604 are noted in the right column.

FIG. 4A and FIG. 4B: Immune gene transcripts are differentially regulated in an HPV6 specific fashion after treatment with INO-3106. Heat maps showing fold difference of differentially expressed genes in stimulated versus unstimulated cells pre and post vaccination. (FIG. 4A) Fold change in gene expression (>2 fold) in cells stimulated with peptide pool versus medium alone for 24 hours. (FIG. 4B) Fold change in gene expression (>2 fold) after 11 days of T cell expansion followed by restimulation of cells with peptide pool versus medium alone for 24 hours. Data are transformed to log 2 fold change, with red indicating upregulation and green indicating downregulation.

FIG. 5: Treatment of RRP patients with INO-3106 imparts clinical benefit in the form of avoidance of surgery. Top panel—Swimmers plot indicating the length of time in Days that subjects 604 and 603 were surgery-free. The dotted red line indicates the timepoint surgery would be expected based on previous surgery frequencies prior to intervention with INO-3106. D indicates the timepoint at which subject 604 required surgery. 2\, indicates that as of the indicated timepoint subject 603 remains surgery free. Bottom left panel—Green bars track to the left y-axis and indicate the magnitude of HPV6-specific CD8+ T cells expressing CD38, Ki67, Granzyme A, Granzyme B and Perforin. Blue bars track to the right y-axis and indicate the fold-change in surgery-free time relative to the expected surgery frequencies for these subjects. Bottom right—the chart indicates patient ID, fold increase in surgery free time, total surgery free time and the increase in surgery free time experienced by these subjects after treatment with INO-3106.

FIG. 6 depicts the results of experiments assessing the HPV6 E6 and E7 cellular immune responses for subject 601.

### DETAILED DESCRIPTION OF PREFERRED EMBODIMENTS

Definitions.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6,9, and 7.0 are explicitly contemplated.

a. Adjuvant

“Adjuvant” as used herein may mean any molecule added to the DNA plasmid vaccines described herein to enhance antigenicity of the one or more antigens encoded by the DNA plasmids and encoding nucleic acid sequences described hereinafter.

b. Antibody

“Antibody” may mean an antibody of classes IgG, IgM, IgA, IgD or IgE, or fragments, fragments or derivatives thereof, including Fab, F(ab′)2, Fd, and single chain antibodies, diabodies, bispecific antibodies, bifunctional antibodies and derivatives thereof. The antibody may be an antibody isolated from the serum sample of mammal, a polyclonal antibody, affinity purified antibody, or mixtures thereof which exhibits sufficient binding specificity to a desired epitope or a sequence derived therefrom.

c. Antigen

“Antigen” refers to: proteins having an HPV E6 or HPV E7 domain, and preferably and E6 and E7 fusion with an endeoproteolytic cleavage site therebetween. Antigens include SEQ ID NOs: 2 (subtype 6); fragments thereof of lengths set forth herein, variants, i.e. proteins with sequences homologous to SEQ ID NO:2 as set forth herein, fragments of variants having lengths set forth herein, and combinations thereof. Antigens may have an IgE leader sequence of SEQ ID NO:4 or may alternatively have such sequence removed from the N-terminal end. Antigens may optionally include signal peptides such as those from other proteins.

d. Coding Sequence

“Coding sequence” or “encoding nucleic acid” as used herein may mean refers to the nucleic acid (RNA or DNA molecule) that comprise a nucleotide sequence which encodes an antigen as set forth in section c. above. The coding sequence may further include initiation and termination signals operably linked to regulatory elements including a promoter and polyadenylation signal capable of directing expression in the cells of an individual or mammal to whom the nucleic acid is administered. The coding sequence may further include sequences that encode signal peptides, e.g., an IgE leader sequence such as SEQ ID NO:3.

e. Complement

“Complement” or “complementary” as used herein may mean a nucleic acid may mean Watson-Crick (e.g., A-T/U and C-G) or Hoogsteen base pairing between nucleotides or nucleotide analogs of nucleic acid molecules.

f. Fragment

“Fragment” may mean a polypeptide fragment of an antigen that is capable of eliciting an immune response in a mammal against the antigen. A fragment of an antigen may be 100% identical to the full length except missing at least one amino acid from the N and/or C terminal, in each case with or without signal peptides and/or a methionine at position 1. Fragments may comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length antigen, excluding any heterologous signal peptide added. The fragment may, preferably, comprise a fragment of a polypeptide that is 95% or more, 96% or more, 97% or more, 98% or more or 99% or more homologous to the antigen and additionally comprise an N terminal methionine or heterologous signal peptide which is not included when calculating percent homology Fragments may further comprise an N terminal methionine and/or a signal peptide such as an immunoglobulin signal peptide, for example an IgE or IgG signal peptide. The N terminal methionine and/or signal peptide may be linked to a fragment of an antigen.

A fragment of a nucleic acid sequence that encodes antigen may be 100% identical to the full length except missing at least one nucleotide from the 5′ and/or 3′ end, in each case with or without sequences encoding signal peptides and/or a methionine at position 1. Fragments may comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the particular full length coding sequence, excluding any heterologous signal peptide added. The fragment may, preferably, comprise a fragment that encodes a polypeptide that is 95% or more, 96% or more, 97% or more, 98% or more or 99% or more homologous to the antigen and additionally optionally comprise sequence encoding an N terminal methionine or heterologous signal peptide which is not included when calculating percent homology Fragments may further comprise coding sequences for an N terminal methionine and/or a signal peptide such as an immunoglobulin signal peptide, for example an IgE or IgG signal peptide. The coding sequence encoding the N terminal methionine and/or signal peptide may be linked to a fragment of coding sequence.

g. Identical

“Identical” or “identity” as used herein in the context of two or more nucleic acids or polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0.

h. Immune Response

“Immune response” as used herein may mean the activation of a host's immune system, e.g., that of a mammal, in response to the introduction of one or more antigens via the provided DNA plasmid vaccines. The immune response can be in the form of a cellular or humoral response, or both.

i. Nucleic Acid

“Nucleic acid” or “oligonucleotide” or “polynucleotide” as used herein may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

j. Operably Linked

“Operably linked” as used herein may mean that expression of a gene is under the control of a promoter with which it is spatially connected. A promoter may be positioned 5′ (upstream) or 3′ (downstream) of a gene under its control. The distance between the promoter and a gene may be approximately the same as the distance between that promoter and the gene it controls in the gene from which the promoter is derived. As is known in the art, variation in this distance may be accommodated without loss of promoter function.

k. Promoter

“Promoter” as used herein may mean a synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of same. A promoter may also comprise distal enhancer or repressor elements, which can be located as much as several thousand base pairs from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter and the CMV IE promoter.

1. Stringent Hybridization Conditions

“Stringent hybridization conditions” as used herein may mean conditions under which a first nucleic acid sequence (e.g., probe) will hybridize to a second nucleic acid sequence (e.g., target), such as in a complex mixture of nucleic acids. Stringent conditions are sequence-dependent and will be different in different circumstances. Stringent conditions may be selected to be about 5 10° C. lower than the thermal melting point (Tm) for the specific sequence at a defined ionic strength pH. The Tm may be the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions may be those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions may also be achieved with the addition of destabilizing agents such as formamide. For selective or specific hybridization, a positive signal may be at least 2 to 10 times background hybridization. Exemplary stringent hybridization conditions include the following: 50% formamide, 5×SSC, and 1% SDS, incubating at 42° C., or, 5×SSC, 1% SDS, incubating at 65° C., with wash in 0.2×SSC, and 0.1% SDS at 65° C.

m. Substantially Complementary

“Substantially complementary” as used herein may mean that a first sequence is at least 60%, 65%, 70%, 75%, 80%, 85%, 90%, 95%, 97%, 98% or 99% identical to the complement of a second sequence over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or that the two sequences hybridize under stringent hybridization conditions.

n. Substantially Identical

“Substantially identical” as used herein may mean that a first and second sequence are at least 60%, 65%, 70%, 75%, 80%, 85%, 90%, 95%, 97%, 98% or 99% identical over a region of 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100 or more nucleotides or amino acids, or with respect to nucleic acids, if the first sequence is substantially complementary to the complement of the second sequence.

o. Variant

“Variant” used herein with respect to a nucleic acid may mean (i) a portion or fragment of a referenced nucleotide sequence; (ii) the complement of a referenced nucleotide sequence or portion thereof; (iii) a nucleic acid that is substantially identical to a referenced nucleic acid or the complement thereof; or (iv) a nucleic acid that hybridizes under stringent conditions to the referenced nucleic acid, complement thereof, or a sequences substantially identical thereto.

“Variant” with respect to a peptide or polypeptide that differs in amino acid sequence by the insertion, deletion, or conservative substitution of amino acids, but retain at least one biological activity. Variant may also mean a protein with an amino acid sequence that is substantially identical to a referenced protein with an amino acid sequence that retains at least one biological activity. A conservative substitution of an amino acid, i.e., replacing an amino acid with a different amino acid of similar properties (e.g., hydrophilicity, degree and distribution of charged regions) is recognized in the art as typically involving a minor change. These minor changes can be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids can also be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity. U.S. Pat. No. 4,554,101, incorporated fully herein by reference. Substitution of amino acids having similar hydrophilicity values can result in peptides retaining biological activity, for example immunogenicity, as is understood in the art. Substitutions may be performed with amino acids having hydrophilicity values within ±2 of each other. Both the hyrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

p. Vector

“Vector” used herein may mean a nucleic acid sequence containing an origin of replication. A vector may be a plasmid, bacteriophage, bacterial artificial chromosome or yeast artificial chromosome. A vector may be a DNA or RNA vector. A vector may be either a self-replicating extrachromosomal vector or a vector which integrates into a host genome.

Improved vaccines are disclosed which arise from a multi-phase strategy to enhance cellular immune responses induced by immunogens. Modified consensus sequences were generated. Genetic modifications including codon optimization, RNA optimization, and the addition of a high efficient immunoglobin leader sequence are also disclosed. The novel construct has been designed to elicit stronger and broader cellular immune responses than a corresponding codon optimized immunogens.

The improved HPV vaccines are based upon proteins and genetic constructs that encode proteins with epitopes that make them particularly effective as immunogens, such that they mediate a prophylactic or therapeutic strategy against RRP. Accordingly, vaccines may induce a therapeutic or prophylactic immune response. In some embodiments, the means to deliver the immunogen is a DNA vaccine, a recombinant vaccine, a protein subunit vaccine, a composition comprising the immunogen, an attenuated vaccine or a killed vaccine. In some embodiments, the vaccine comprises a combination selected from the groups consisting of: one or more DNA vaccines, one or more recombinant vaccines, one or more protein subunit vaccines, one or more compositions comprising the immunogen, one or more attenuated vaccines and one or more killed vaccines.

According to some embodiments, a vaccine is delivered to an individual to modulate the activity of the individual's immune system and thereby enhance the immune response against HPV to treat RRP. When a nucleic acid molecule that encodes the protein is taken up by cells of the individual the nucleotide sequence is expressed in the cells and the protein are thereby delivered to the individual. Methods of delivering the coding sequences of the protein on nucleic acid molecule such as plasmid, as part of recombinant vaccines and as part of attenuated vaccines, as isolated proteins or proteins part of a vector are provided.

Compositions and methods are provided which provide a prophylactic and/or therapeutic treatment against RRP in an individual.

Compositions for delivering nucleic acid molecules that comprise a nucleotide sequence that encodes the immunogen are operably linked to regulatory elements. Compositions may include a plasmid that encodes the immunogen, a recombinant vaccine comprising a nucleotide sequence that encodes the immunogen, a live attenuated pathogen that encodes a protein of the invention and/or includes a protein of the invention; a killed pathogen includes a protein of the invention; or a composition such as a liposome or subunit vaccine that comprises a protein of the invention. The present invention further relates to injectable pharmaceutical compositions that comprise compositions.

Aspects of the invention provide compositions comprising at least one nucleotide sequence comprising an HPV6 E6-E7 fusion antigen.

Another aspect provides compositions comprising one or more nucleotide sequences encoding an HPV6 E6-E7 fusion antigen selected from the group consisting of: nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; a nucleotide sequence that is at least 95% homologous to a fragment of a nucleotide sequence that encodes SEQ ID NO:2.

In some embodiments the compositions include HPV6 E6-E7 fusion antigens selected from the group consisting of: nucleotide sequence that encodes SEQ ID NO:2; a nucleotide sequence that is at least 95% homologous to a nucleotide sequence that encodes SEQ ID NO:2; a fragment of a nucleotide sequence that encodes SEQ ID NO:2; a nucleotide sequence that is at least 95% homologous to a fragment of a nucleotide sequence that encodes SEQ ID NO:2.

In another aspect of the invention, there are provided compositions comprising one or more nucleotide sequences encoding an HPV6 E6-E7 fusion antigen selected from the group consisting of: SEQ ID NO:1; a nucleotide sequence that is at least 95% homologous to SEQ ID NO:1; a fragment of SEQ ID NO:1; a nucleotide sequence that is at least 95% homologous to a fragment of SEQ ID NO:1.

In some embodiments the nucleotide sequences described herein is absent the leader sequence. In one embodiment, the nucleotide sequences comprising HPV6 E6-E7 fusion antigen is absent a leader sequence. In particular, the HPV6 E6-E7 fusion antigens including nucleotide sequence that encodes SEQ ID NO:2; are absent a leader sequence at 5′ end, for example nucleotide sequence encoding SEQ ID NO:4. In particular, the HPV6 E6-E7 fusion antigens including nucleotide sequence SEQ ID NO:1 are absent a leader sequence at 5′ end, for example nucleotide sequence SEQ ID NO:3.

In some embodiments the nucleotide sequences of the present invention can be 80%, 81%, 82%, 83%, 84%, 85%, 86%, 87%, 88%, 89%, 90%, 91%, 92%, 93%, 94%, 95%, 96%, 97%, 98%, or 99% homologous with the provided nucleotide sequences; preferably 95%, 96%, 97%, 98%, or 99%; or 98% or 99%.

The nucleotide sequences provided can be included into one of a variety of known vectors or delivery systems, including a plasmid, viral vector, lipid vector, nanoparticle; preferably a plasmid.

In additional aspects, provided are pharmaceutical compositions comprising the disclosed nucleotide sequences.

In some aspects, there are methods of inducing an effective immune response in an individual against more than one subtype of HPV thereby providing a prophylactic or therapeutic treatment against RRP, comprising administering to said individual a composition comprising one or more of the nucleotides sequences provided; preferably, the compositions have more than one antigen. The methods preferably include a step of introducing the provided nucleotide sequences into the individual by electroporation.

SEQ ID NO:1 comprises a nucleotide sequence that encodes a consensus immunogen of HPV6 E6 and E7 proteins. SEQ ID NO:1 includes an IgE leader sequence SEQ ID NO:3 linked to the nucleotide sequence at the 5′ end of SEQ ID NO:1. SEQ ID NO:2 comprises the amino acid sequence for the consensus immunogen of HPV 6 E6 and E7 proteins. SEQ ID NO:2 includes an IgE leader sequence SEQ ID NO:4 at the N-terminal end of the consensus immunogen sequence. The IgE leader sequence is SEQ ID NO:4 and can be encoded by SEQ ID NO:3. Further information regarding the HPV6 E6-E7 fusion antigen can be found at least in U.S. Pat. No. 9,050,287, which is incorporated by reference in its entirety.

In some embodiments, vaccines include SEQ ID NO:2, or a nucleic acid molecule that encodes SEQ ID NO:2.

Fragments of SEQ ID NO:2 may be 100% identical to the full length except missing at least one amino acid from the N and/or C terminal, in each case with or without signal peptides and/or a methionine at position 1. Fragments of SEQ ID NO:2 can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the full length SEQ ID NO:2, excluding any heterologous signal peptide added. The fragment can, preferably, comprise a fragment of SEQ ID NO:2 that is 95% or more, 96% or more, 97% or more, 98% or more or 99% or more homologous to SEQ ID NO:2 and additionally comprise an N terminal methionine or heterologous signal peptide which is not included when calculating percent homology Fragments can further comprise an N terminal methionine and/or a signal peptide such as an immunoglobulin signal peptide, for example an IgE or IgG signal peptide. The N terminal methionine and/or signal peptide may be linked to the fragment.

Fragments of a nucleic acid sequence SEQ ID NO:1 can be 100% identical to the full length except missing at least one nucleotide from the 5′ and/or 3′ end, in each case with or without sequences encoding signal peptides and/or a methionine at position 1. Fragments can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of full length coding sequence SEQ ID NO:1, excluding any heterologous signal peptide added. The fragment can, preferably, comprise a fragment that encodes a polypeptide that is 95% or more, 96% or more, 97% or more, 98% or more or 99% or more homologous to the antigen SEQ ID NO:2 and additionally optionally comprise sequence encoding an N terminal methionine or heterologous signal peptide which is not included when calculating percent homology Fragments can further comprise coding sequences for an N terminal methionine and/or a signal peptide such as an immunoglobulin signal peptide, for example an IgE or IgG signal peptide. The coding sequence encoding the N terminal methionine and/or signal peptide may be linked to the fragment.

In some embodiments, fragments of SEQ ID NO:1 may comprise 786 or more nucleotides; in some embodiments, 830 or more nucleotides; in some embodiments 856 or more nucleotides; and in some embodiments, 865 or more nucleotides. In some embodiments, fragments of SEQ ID NO:1 such as those set forth herein may further comprise coding sequences for the IgE leader sequences. In some embodiments, fragments of SEQ ID NO:1 do not comprise coding sequences for the IgE leader sequences.

In some embodiments, fragments of SEQ ID NO:2 may comprise 252 or more amino acids; in some embodiments, 266 or more amino acids; in some embodiments, 275 or more amino acids; and in some embodiments, 278 or more amino acids.

In one embodiment, the HPV6 E6-E7 immunogen or nucleic acid molecule encoding the HPV6 E6-E7 immunogen is administered in combination with IL-12. In one embodiment, IL-12 is encoded from a synthetic DNA plasmid.

In some embodiments, the method comprises administering a composition comprising a nucleic acid molecule encoding the p35 and/or p40 subunit of IL-12.

SEQ ID NO:5 comprises a nucleotide sequence that encodes p35 subunit of IL-12. SEQ ID NO:6 comprises the amino acid sequence for p35 subunit of IL-12.

In some embodiments, vaccines include SEQ ID NO:6, or a nucleic acid molecule that encodes SEQ ID NO:6.

Fragments of SEQ ID NO:6 may be 100% identical to the full length except missing at least one amino acid from the N and/or C terminal, in each case with or without signal peptides and/or a methionine at position 1. Fragments of SEQ ID NO:6 can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the full length SEQ ID NO:6, excluding any heterologous signal peptide added. The fragment can, preferably, comprise a fragment of SEQ ID NO:6 that is 95% or more, 96% or more, 97% or more, 98% or more or 99% or more homologous to SEQ ID NO:6 and additionally comprise an N terminal methionine or heterologous signal peptide which is not included when calculating percent homology. Fragments can further comprise an N terminal methionine and/or a signal peptide such as an immunoglobulin signal peptide, for example an IgE or IgG signal peptide. The N terminal methionine and/or signal peptide may be linked to the fragment.

Fragments of a nucleic acid sequence SEQ ID NO:5 can be 100% identical to the full length except missing at least one nucleotide from the 5′ and/or 3′ end, in each case with or without sequences encoding signal peptides and/or a methionine at position 1. Fragments can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of full length coding sequence SEQ ID NO:5, excluding any heterologous signal peptide added. The fragment can, preferably, comprise a fragment that encodes a polypeptide that is 95% or more, 96% or more, 97% or more, 98% or more or 99% or more homologous to the antigen SEQ ID NO:6 and additionally optionally comprise sequence encoding an N terminal methionine or heterologous signal peptide which is not included when calculating percent homology. Fragments can further comprise coding sequences for an N terminal methionine and/or a signal peptide such as an immunoglobulin signal peptide, for example an IgE or IgG signal peptide. The coding sequence encoding the N terminal methionine and/or signal peptide may be linked to the fragment.

In some embodiments, fragments of SEQ ID NO:5 may comprise 500 or more nucleotides; in some embodiments, 550 or more nucleotides; in some embodiments 600 or more nucleotides; and in some embodiments, 630 or more nucleotides. In some embodiments, fragments of SEQ ID NO:5 such as those set forth herein may further comprise coding sequences for the IgE leader sequences. In some embodiments, fragments of SEQ ID NO:5 do not comprise coding sequences for the IgE leader sequences.

In some embodiments, fragments of SEQ ID NO:6 may comprise 150 or more amino acids; in some embodiments, 175 or more amino acids; in some embodiments, 200 or more amino acids; and in some embodiments, 210 or more amino acids.

SEQ ID NO:7 comprises a nucleotide sequence that encodes p40 subunit of IL-12. SEQ ID NO:8 comprises the amino acid sequence for p35 subunit of IL-12.

In some embodiments, vaccines include SEQ ID NO:8, or a nucleic acid molecule that encodes SEQ ID NO:8.

Fragments of SEQ ID NO:8 may be 100% identical to the full length except missing at least one amino acid from the N and/or C terminal, in each case with or without signal peptides and/or a methionine at position 1. Fragments of SEQ ID NO:8 can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of the full length SEQ ID NO:8, excluding any heterologous signal peptide added. The fragment can, preferably, comprise a fragment of SEQ ID NO:8 that is 95% or more, 96% or more, 97% or more, 98% or more or 99% or more homologous to SEQ ID NO:8 and additionally comprise an N terminal methionine or heterologous signal peptide which is not included when calculating percent homology. Fragments can further comprise an N terminal methionine and/or a signal peptide such as an immunoglobulin signal peptide, for example an IgE or IgG signal peptide. The N terminal methionine and/or signal peptide may be linked to the fragment.

Fragments of a nucleic acid sequence SEQ ID NO:7 can be 100% identical to the full length except missing at least one nucleotide from the 5′ and/or 3′ end, in each case with or without sequences encoding signal peptides and/or a methionine at position 1. Fragments can comprise 60% or more, 65% or more, 70% or more, 75% or more, 80% or more, 85% or more, 90% or more, 91% or more, 92% or more, 93% or more, 94% or more, 95% or more, 96% or more, 97% or more, 98% or more, 99% or more percent of the length of full length coding sequence SEQ ID NO:7, excluding any heterologous signal peptide added. The fragment can, preferably, comprise a fragment that encodes a polypeptide that is 95% or more, 96% or more, 97% or more, 98% or more or 99% or more homologous to the antigen SEQ ID NO:8 and additionally optionally comprise sequence encoding an N terminal methionine or heterologous signal peptide which is not included when calculating percent homology. Fragments can further comprise coding sequences for an N terminal methionine and/or a signal peptide such as an immunoglobulin signal peptide, for example an IgE or IgG signal peptide. The coding sequence encoding the N terminal methionine and/or signal peptide may be linked to the fragment.

In some embodiments, fragments of SEQ ID NO:7 may comprise 850 or more nucleotides; in some embodiments, 900 or more nucleotides; in some embodiments 930 or more nucleotides; and in some embodiments, 960 or more nucleotides. In some embodiments, fragments of SEQ ID NO:7 such as those set forth herein may further comprise coding sequences for the IgE leader sequences. In some embodiments, fragments of SEQ ID NO:7 do not comprise coding sequences for the IgE leader sequences.

In some embodiments, fragments of SEQ ID NO:8 may comprise 250 or more amino acids; in some embodiments, 275 or more amino acids; in some embodiments, 300 or more amino acids; and in some embodiments, 315 or more amino acids.

In some embodiments, the method comprises concurrent administration of: (a) a composition comprising a nucleic acid molecule encoding an HPV6 antigen disclosed herein (e.g. HPV6 E6-E7 fusion antigen) and (b) a composition comprising a nucleic acid molecule encoding one or more IL-12 subunit (e.g. p35 and/or p40) disclosed herein. In some embodiments, the method comprises administering a composition comprising a nucleic acid molecule encoding one or more IL-12 subunit (e.g. p35 and/or p40) disclosed herein after the prior administration of a composition comprising a nucleic acid molecule encoding an HPV6 antigen disclosed herein (e.g. HPV6 E6-E7 fusion antigen). In some embodiments, the method comprises administering a composition comprising a nucleic acid molecule encoding an HPV6 antigen disclosed herein (e.g. HPV6 E6-E7 fusion antigen) after the prior administration of a composition comprising a nucleic acid molecule encoding one or more IL-12 subunit (e.g. p35 and/or p40) disclosed herein.

Methods of treating or preventing RRP in a subject by inducing an immune response in an individual against HPV comprising administering to said individual a composition comprising a nucleic acid sequences provided herein. In some embodiments, the methods also include introducing the nucleic acid sequences into the individual by electroporation.

In some aspects, there are methods of treating or preventing RRP in a subject by inducing an immune response in an individual against HPV comprising administering to said individual a composition comprising a amino acid sequence provided herein. In some embodiments, the methods also include introducing the amino acid sequences into the individual by electroporation.

Improved vaccines comprise proteins and genetic constructs that encode proteins with epitopes that make them particularly effective as immunogens against which anti-HPV immune responses can be induced. Accordingly, vaccines can be provided to induce a therapeutic or prophylactic immune response. In some embodiments, the means to deliver the immunogen is a DNA vaccine, a recombinant vaccine, a protein subunit vaccine, a composition comprising the immunogen, an attenuated vaccine or a killed vaccine. In some embodiments, the vaccine comprises a combination selected from the groups consisting of: one or more DNA vaccines, one or more recombinant vaccines, one or more protein subunit vaccines, one or more compositions comprising the immunogen, one or more attenuated vaccines and one or more killed vaccines.

Aspects of the invention provide methods of delivering the coding sequences of the protein on nucleic acid molecule such as plasmid, as part of recombinant vaccines and as part of attenuated vaccines, as isolated proteins or proteins part of a vector.

According to some aspects of the present invention, compositions and methods are provided which prophylactically and/or therapeutically immunize an individual.

DNA vaccines are described in U.S. Pat. Nos. 5,593,972, 5,739,118, 5,817,637, 5,830,876, 5,962,428, 5,981,505, 5,580,859, 5,703,055, 5,676,594, and the priority applications cited therein, which are each incorporated herein by reference. In addition to the delivery protocols described in those applications, alternative methods of delivering DNA are described in U.S. Pat. Nos. 4,945,050 and 5,036,006, which are both incorporated herein by reference.

The present invention relates to improved attenuated live vaccines, improved killed vaccines and improved vaccines that use recombinant vectors to deliver foreign genes that encode antigens and well as subunit and glycoprotein vaccines. Examples of attenuated live vaccines, those using recombinant vectors to deliver foreign antigens, subunit vaccines and glycoprotein vaccines are described in U.S. Pat. Nos. 4,510,245; 4,797,368; 4,722,848; 4,790,987; 4,920,209; 5,017,487; 5,077,044; 5,110,587; 5,112,749; 5,174,993; 5,223,424; 5,225,336; 5,240,703; 5,242,829; 5,294,441; 5,294,548; 5,310,668; 5,387,744; 5,389,368; 5,424,065; 5,451,499; 5,453,364; 5,462,734; 5,470,734; 5,474,935; 5,482,713; 5,591,439; 5,643,579; 5,650,309; 5,698,202; 5,955,088; 6,034,298; 6,042,836; 6,156,319 and 6,589,529, which are each incorporated herein by reference.

When taken up by a cell, the genetic construct(s) may remain present in the cell as a. functioning extrachromosomal molecule and/or integrate into the cell's chromosomal DNA. DNA may be introduced into cells where it remains as separate genetic material in the form of a plasmid or plasmids. Alternatively, linear DNA that can integrate into the chromosome may be introduced into the cell. When introducing DNA into the cell, reagents that promote DNA integration into chromosomes may be added. DNA sequences that are useful to promote integration may also be included in the DNA molecule. Alternatively, RNA may be administered to the cell. It is also contemplated to provide the genetic construct as a linear minichromosome including a centromere, telomeres and an origin of replication. Gene constructs may remain part of the genetic material in attenuated live microorganisms or recombinant microbial vectors which live in cells. Gene constructs may be part of genomes of recombinant viral vaccines where the genetic material either integrates into the chromosome of the cell or remains extrachromosomal. Genetic constructs include regulatory elements necessary for gene expression of a nucleic acid molecule. The elements include: a promoter, an initiation codon, a stop codon, and a polyadenylation signal. In addition, enhancers are often required for gene expression of the sequence that encodes the target protein or the immunomodulating protein. It is necessary that these elements be operable linked to the sequence that encodes the desired proteins and that the regulatory elements are operably in the individual to whom they are administered.

Initiation codons and stop codon are generally considered to be part of a nucleotide sequence that encodes the desired protein. However, it is necessary that these elements are functional in the individual to whom the gene construct is administered. The initiation and termination codons must be in frame with the coding sequence.

Promoters and polyadenylation signals used must be functional within the cells of the individual.

Examples of promoters useful to practice the present invention, especially in the production of a genetic vaccine for humans, include but are not limited to promoters from Simian Virus 40 (SV40), Mouse Mammary Tumor Virus (MMTV) promoter, Human Immunodeficiency Virus (MV) such as the BIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human Actin, human Myosin, human Hemoglobin, human muscle creatine and human metalothionein.

Examples of polyadenylation signals useful to practice the present invention, especially in the production of a genetic vaccine for humans, include but are not limited to SV40 polyadenylation signals and LTR polyadenylation signals. In particular, the SV40 polyadenylation signal that is in pCEP4 plasmid (Invitrogen, San Diego Calif.), referred to as the SV40 polyadenylation signal, is used.

In addition to the regulatory elements required for DNA expression, other elements may also be included in the DNA molecule. Such additional elements include enhancers. The enhancer may be selected from the group including but not limited to: human Actin, human Myosin, human Hemoglobin, human muscle creatine and viral enhancers such as those from CMV, RSV and EBV.

Genetic constructs can be provided with mammalian origin of replication in order to maintain the construct extrachromosomally and produce multiple copies of the construct in the cell. Plasmids pVAX1, pCEP4 and pREP4 from Invitrogen (San Diego, Calif.) contain the Epstein Barr virus origin of replication and nuclear antigen EBNA-1 coding region which produces high copy episomal replication without integration.

In some preferred embodiments related to immunization applications, nucleic acid molecule(s) are delivered which include nucleotide sequences that encode protein of the invention, and, additionally, genes for proteins which further enhance the immune response against such target proteins. Examples of such genes are those which encode other cytokines and lymphokines such as alpha-interferon, gamma-interferon, platelet derived growth factor (PDGF), TNFα, TNFβ, GM-CSF, epidermal growth factor (EGF), IL-1, IL-2, IL-4, IL-5, IL-6, IL-10, IL-12, IL-18, MHC, CD80, CD86 and IL-15 including IL-15 having the signal sequence deleted and optionally including the signal peptide from IgE. Other genes which may be useful include those encoding: MCP-1, MIP-la, MIP-1p, IL-8, RANTES, L-selectin, P-selectin, E-selectin, CD34, GlyCAM-1, MadCAM-1, LFA-1, VLA-1, Mac-1, p150.95, PECAM, ICAM-1, ICAM-2, ICAM-3, CD2, LFA-3, M-CSF, G-CSF, IL-4, mutant forms of IL-18, CD40, CD40L, vascular growth factor, IL-7, nerve growth factor, vascular endothelial growth factor, Fas, TNF receptor, Flt, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IkB, Inactive NIK, SAP K, SAP-1, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof

An additional element may be added which serves as a target for cell destruction if it is desirable to eliminate cells receiving the genetic construct for any reason. A herpes thymidine kinase (tk) gene in an expressible form can be included in the genetic construct. The drug gangcyclovir can be administered to the individual and that drug will cause the selective killing of any cell producing tk, thus, providing the means for the selective destruction of cells with the genetic construct.

In order to maximize protein production, regulatory sequences may be selected which are well suited for gene expression in the cells the construct is administered into. Moreover, codons may be selected which are most efficiently transcribed in the cell. One having ordinary skill in the art can produce DNA constructs that are functional in the cells.

In some embodiments, gene constructs may be provided in which the coding sequences for the proteins described herein are linked to IgE signal peptide. In some embodiments, proteins described herein are linked to IgE signal peptide.

In some embodiments for which protein is used, for example, one having ordinary skill in the art can, using well known techniques, produce and isolate proteins of the invention using well known techniques. In some embodiments for which protein is used, for example, one having ordinary skill in the art can, using well known techniques, inserts DNA molecules that encode a protein of the invention into a commercially available expression vector for use in well known expression systems. For example, the commercially available plasmid pSE420 (Invitrogen, San Diego, Calif.) may be used for production of protein in E. coli. The commercially available plasmid pYES2 (Invitrogen, San Diego, Calif.) may, for example, be used for production in S. cerevisiae strains of yeast. The commercially available MAXBAC™ complete baculovirus expression system (Invitrogen, San Diego, Calif.) may, for example, be used for production in insect cells. The commercially available plasmid pcDNA I or pcDNA3 (Invitrogen, San Diego, Calif.) may, for example, be used for production in mammalian cells such as Chinese Hamster Ovary cells. One having ordinary skill in the art can use these commercial expression vectors and systems or others to produce protein by routine techniques and readily available starting materials. (See e.g., Sambrook et al., Molecular Cloning a Laboratory Manual, Second Ed. Cold Spring Harbor Press (1989) which is incorporated herein by reference.) Thus, the desired proteins can be prepared in both prokaryotic and eukaryotic systems, resulting in a spectrum of processed forms of the protein.

One having ordinary skill in the art may use other commercially available expression vectors and systems or produce vectors using well known methods and readily available starting materials. Expression systems containing the requisite control sequences, such as promoters and polyadenylation signals, and preferably enhancers are readily available and known in the art for a variety of hosts. See e.g., Sambrook et al., Molecular Cloning a Laboratory Manual, Second Ed. Cold Spring Harbor Press (1989). Genetic constructs include the protein coding sequence operably linked to a promoter that is functional in the cell line into which the constructs are transfected. Examples of constitutive promoters include promoters from cytomegalovirus or SV40. Examples of inducible promoters include mouse mammary leukemia virus or metallothionein promoters. Those having ordinary skill in the art can readily produce genetic constructs useful for transfecting with cells with DNA that encodes protein of the invention from readily available starting materials. The expression vector including the DNA that encodes the protein is used to transform the compatible host which is then cultured and maintained under conditions wherein expression of the foreign DNA takes place.

The protein produced is recovered from the culture, either by lysing the cells or from the culture medium as appropriate and known to those in the art. One having ordinary skill in the art can, using well known techniques, isolate protein that is produced using such expression systems. The methods of purifying protein from natural sources using antibodies which specifically bind to a specific protein as described above may be equally applied to purifying protein produced by recombinant DNA methodology.

In addition to producing proteins by recombinant techniques, automated peptide synthesizers may also be employed to produce isolated, essentially pure protein. Such techniques are well known to those having ordinary skill in the art and are useful if derivatives which have substitutions not provided for in DNA-encoded protein production.

The nucleic acid molecules may be delivered using any of several well known technologies including DNA injection (also referred to as DNA vaccination), recombinant vectors such as recombinant adenovirus, recombinant adenovirus associated virus and recombinant vaccinia.

Routes of administration include, but are not limited to, intramuscular, intransally, intraperitoneal, intradermal, subcutaneous, intravenous, intraarterially, intraoccularly and oral as well as topically, transdermally, by inhalation or suppository or to mucosal tissue such as by lavage to vaginal, rectal, urethral, buccal and sublingual tissue. Preferred routes of administration include intramuscular, intraperitoneal, intradermal and subcutaneous injection. Genetic constructs may be administered by means including, but not limited to, electroporation methods and devices, traditional syringes, needleless injection devices, or “microprojectile bombardment gone guns”.

Examples of electroporation devices and electroporation methods preferred for facilitating delivery of the DNA vaccines, include those described in U.S. Pat. No. 7,245,963 by Draghia-Akli, et al., U.S. Patent Pub. 2005/0052630 submitted by Smith, et al., the contents of which are hereby incorporated by reference in their entirety. Also preferred, are electroporation devices and electroporation methods for facilitating delivery of the DNA vaccines provided in co-pending and co-owned U.S. patent application Ser. No. 11/874,072, filed Oct. 17, 2007, which claims the benefit under 35 USC 119(e) to U.S. Provisional Application Ser. No. 60/852,149, filed Oct. 17, 2006, and 60/978,982, filed Oct. 10, 2007, all of which are hereby incorporated in their entirety.

The following is an example of an embodiment using electroporation technology, and is discussed in more detail in the patent references discussed above: electroporation devices can be configured to deliver to a desired tissue of a mammal a pulse of energy producing a constant current similar to a preset current input by a user. The electroporation device comprises an electroporation component and an electrode assembly or handle assembly. The electroporation component can include and incorporate one or more of the various elements of the electroporation devices, including: controller, current waveform generator, impedance tester, waveform logger, input element, status reporting element, communication port, memory component, power source, and power switch. The electroporation component can function as one element of the electroporation devices, and the other elements are separate elements (or components) in communication with the electroporation component. In some embodiments, the electroporation component can function as more than one element of the electroporation devices, which can be in communication with still other elements of the electroporation devices separate from the electroporation component. The use of electroporation technology to deliver the improved HPV vaccine is not limited by the elements of the electroporation devices existing as parts of one electromechanical or mechanical device, as the elements can function as one device or as separate elements in communication with one another. The electroporation component is capable of delivering the pulse of energy that produces the constant current in the desired tissue, and includes a feedback mechanism. The electrode assembly includes an electrode array having a plurality of electrodes in a spatial arrangement, wherein the electrode assembly receives the pulse of energy from the electroporation component and delivers same to the desired tissue through the electrodes. At least one of the plurality of electrodes is neutral during delivery of the pulse of energy and measures impedance in the desired tissue and communicates the impedance to the electroporation component. The feedback mechanism can receive the measured impedance and can adjust the pulse of energy delivered by the electroporation component to maintain the constant current.

In some embodiments, the plurality of electrodes can deliver the pulse of energy in a decentralized pattern. In some embodiments, the plurality of electrodes can deliver the pulse of energy in the decentralized pattern through the control of the electrodes under a programmed sequence, and the programmed sequence is input by a user to the electroporation component. In some embodiments, the programmed sequence comprises a plurality of pulses delivered in sequence, wherein each pulse of the plurality of pulses is delivered by at least two active electrodes with one neutral electrode that measures impedance, and wherein a subsequent pulse of the plurality of pulses is delivered by a different one of at least two active electrodes with one neutral electrode that measures impedance.

In some embodiments, the feedback mechanism is performed by either hardware or software. Preferably, the feedback mechanism is performed by an analog closed-loop circuit. Preferably, this feedback occurs every 50 μs, 20 μs, 10 μs or 1 μs, but is preferably a real-time feedback or instantaneous (i.e., substantially instantaneous as determined by available techniques for determining response time). In some embodiments, the neutral electrode measures the impedance in the desired tissue and communicates the impedance to the feedback mechanism, and the feedback mechanism responds to the impedance and adjusts the pulse of energy to maintain the constant current at a value similar to the preset current. In some embodiments, the feedback mechanism maintains the constant current continuously and instantaneously during the delivery of the pulse of energy.

In some embodiments, the nucleic acid molecule is delivered to the cells in conjunction with administration of a polynucleotide function enhancer or a genetic vaccine facilitator agent. Polynucleotide function enhancers are described in U.S. Pat. Nos. 5,593,972, 5,962,428 and International Application Serial Number PCT/US94/00899 filed Jan. 26, 1994, which are each incorporated herein by reference. Genetic vaccine facilitator agents are described in U.S. Pat. No. 021,579 filed Apr. 1, 1994, which is incorporated herein by reference. The co-agents that are administered in conjunction with nucleic acid molecules may be administered as a mixture with the nucleic acid molecule or administered separately simultaneously, before or after administration of nucleic acid molecules. In addition, other agents which may function transfecting agents and/or replicating agents and/or inflammatory agents and which may be co-administered with a GVF include growth factors, cytokines and lymphokines such as α-interferon, gamma-interferon, GM-CSF, platelet derived growth factor (PDGF), TNF, epidermal growth factor (EGF), IL-1, IL-2, IL-4, IL-6, IL-10, IL-12 and IL-15 as well as fibroblast growth factor, surface active agents such as immune-stimulating complexes (ISCOMS), Freunds incomplete adjuvant, LPS analog including monophosphoryl Lipid A (WL), muramyl peptides, quinone analogs and vesicles such as squalene and squalene, and hyaluronic acid may also be used administered in conjunction with the genetic construct In some embodiments, an immunomodulating protein may be used as a GVF. In some embodiments, the nucleic acid molecule is provided in association with PLG to enhance delivery/uptake.

The pharmaceutical compositions according to the present invention comprise about 1 nanogram to about 2000 micrograms of DNA. In some preferred embodiments, pharmaceutical compositions according to the present invention comprise about 5 nanogram to about 1000 micrograms of DNA. In some preferred embodiments, the pharmaceutical compositions contain about 10 nanograms to about 800 micrograms of DNA. In some preferred embodiments, the pharmaceutical compositions contain about 0.1 to about 500 micrograms of DNA. In some preferred embodiments, the pharmaceutical compositions contain about 1 to about 350 micrograms of DNA. In some preferred embodiments, the pharmaceutical compositions contain about 25 to about 250 micrograms of DNA. In some preferred embodiments, the pharmaceutical compositions contain about 100 to about 200 microgram DNA.

The pharmaceutical compositions according to the present invention are formulated according to the mode of administration to be used. In cases where pharmaceutical compositions are injectable pharmaceutical compositions, they are sterile, pyrogen free and particulate free. An isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

According to some embodiments of the invention, methods of inducing immune responses are provided. The vaccine may be a protein based, live attenuated vaccine, a cell vaccine, a recombinant vaccine or a nucleic acid or DNA vaccine. In some embodiments, methods of inducing an immune response in individuals against an immunogen, including methods of inducing mucosal immune responses, comprise administering to the individual one or more of CTACK protein, TECK protein, MEC protein and functional fragments thereof or expressible coding sequences thereof in combination with an isolated nucleic acid molecule that encodes protein of the invention and/or a recombinant vaccine that encodes protein of the invention and/or a subunit vaccine that protein of the invention and/or a live attenuated vaccine and/or a killed vaccine. The one or more of CTACK protein, TECK protein, MEC protein and functional fragments thereof may be administered prior to, simultaneously with or after administration of the isolated nucleic acid molecule that encodes an immunogen; and/or recombinant vaccine that encodes an immunogen and/or subunit vaccine that comprises an immunogen and/or live attenuated vaccine and/or killed vaccine. In some embodiments, an isolated nucleic acid molecule that encodes one or more proteins of selected from the group consisting of: CTACK, TECK, MEC and functional fragments thereof is administered to the individual.

The present invention is further illustrated in the following Example. It should be understood that this Example, while indicating embodiments of the invention, is given by way of illustration only. From the above discussion and this Example, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

Each of the U.S. patents, U.S. applications, and references cited throughout this disclosure are hereby incorporated in their entirety by reference.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

Recurrent respiratory papillomatosis (RRP) is a rare disorder characterized by the generation of papillomas of the aerodigestive tract, usually associated with human papilloma virus (HPV) subtypes 6, 11. Current treatment of HPV6 related RRP and invasive malignant diseases could potentially be improved with the addition of HPV-specific immunotherapy. Available preventive HPV vaccines can generate neutralizing antibodies against the HPV major capsid protein L1, but they have not demonstrated therapeutic effects on HPV infection or existing lesions and are unlikely to engender a cytolytic T-cell response (Lin et al., Immunologic research. 2010; 47(1-3):86-112). HPV-specific immunotherapy, on the other hand, may have therapeutic potential to eliminate preexisting lesions and infections by generating immunity against the HPV virus itself and HPV infected cells. HPV E6 and E7 oncoproteins represent ideal targets for this type of therapeutic intervention because of their constitutive expression in HPV associated tumors and their crucial role in the induction and maintenance of HPV associated diseases (Lin et al., Immunologic research. 2010; 47(1-3):86-112).

The experiments presented herein examine the efficacy of INO-3106, a DNA plasmid-based immunotherapy targeting E6 and E7 proteins of HPV6, in order to create a robust immune T cell response in order to treat RRP.

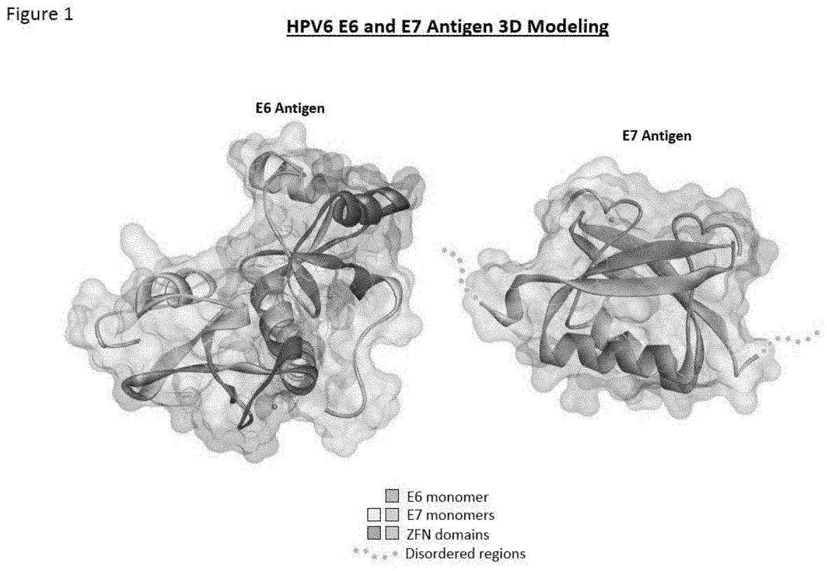

In this study, experiments were conducted to evaluate the efficacy of INO-3106, a novel HPV6-specific immunotherapy consisting of synthetic consensus DNA sequences encoding for HPV6 E6 and E7 (FIG. 1), proteins necessary for HPV6 induced transformation of cancers and tumor maintenance. Synthetic DNA plasmids offer several potential advantages as a immunotherapy platform, including the ability to elicit potent immune responses without evidence of genome integration, a favorable safety profile, stability and relative ease in manufacturing (Saha et al., Recent Pat DNA Gene Seq. 2011; 5(2):92-6). Preclinical studies of INO-3106 have demonstrated strong and specific immune response to HPV6 in animal models (Shin et al., Human vaccines & immunotherapeutics. 2012; 8(4):470-8). HPV16/18-specific therapy (VGX-3100, Inovio Pharmaceuticals, Inc.) designed and evaluated based on the same synthetic consensus platform, has demonstrated cellular immune responses that correlated with clinical benefit in the form of dysplastic lesion regression and elimination of HPV16/18 infection and now support late-phase clinical trials targeting HPV16 and 18 associated diseases (Bagarazzi et al., Sci Transl Med. 2012; 4(155):155ra38).

Preclinical studies have shown that the immunogenicity of DNA vaccines can be substantially increased by the use of cytokine adjuvants (Chattergoon et al., Vaccine. 2004; 22(13-14):1744-50; Hanlon et al., J Virol. 2001; 75(18):8424-33; Kim et al., J Interferon Cytokine Res. 1998; 18(7):537-47; Kim et al., Eur J Immunol. 1998; 28(3):1089-103; Operschall et al., J Clin Virol. 1999; 13(1-2):17-27). Importantly, an engineered plasmid IL-12 genetic adjuvant has been shown to enhance immunogenicity in humans when delivered using the CELLECTRA® device (Kalams et al., Journal of Infectious Diseases. 2013; Tebas et al., The Journal of infectious diseases. 2019). It has been established in multiple clinical studies targeting both skin delivery as well as local muscle that delivery of optimized DNA via the CELLECTRA® device is a highly reproducible method of generating immunity in human beings in a rapid fashion for various purposes ranging from induction prophylactic settings as well as therapeutic approaches (Kalams et al., Journal of Infectious Diseases. 2013; Tebas et al., The Journal of infectious diseases. 2019; Tebas et al., The New England journal of medicine. 2017; Bagarazzi et al., Sci Transl Med. 2012; 4(155):155ra38; Morrow et al., Molecular therapy oncolytics. 2016; 3(16025; Trimble et al., Lancet. 2015; 386(10008):2078-88; Morrow et al., Clinical cancer research: an official journal of the American Association for Cancer Research. 2018; 24(2):276-94; Aggarwal et al., Clinical cancer research: an official journal of the American Association for Cancer Research. 2019; 25(1):110-24; Morrow et al., Molecular therapy: the journal of the American Society of Gene Therapy. 2015; 23(3):591-601).

Here, the present experiments demonstrate the safety and immunogenicity of a pilot study of INO-3106 with or without INO-9012 (IL-12 adjuvant) delivered intramuscularly (IM) via EP with the CELLECTRA® device in patients with HPV6 associated RRP or malignancies. The data from this study suggests that immunotherapy with INO-3106 and IL-12 adjuvant could be a non-invasive immune mediated treatment option for RRP.

In this single-site open-label phase 1 study, subjects with HPV6-positive RRP and malignancies were dosed with INO-3106, a DNA plasmid immunotherapy targeting the E6 and E7 proteins of HPV6, with or without INO-9012, a DNA plasmid immunotherapy that encodes IL-12, delivered intramuscularly (IM) in combination with electroporation (EP) with the CELLECTRA® device. Patients received an escalating dose of INO-3106, 3 mg once and then 6 mg for three additional doses, each dose three weeks apart, with the third and fourth doses co-administered with INO-9012. The primary objective of the study was to evaluate the safety and tolerability of INO-3106 with and without INO-9012. The secondary objective was to determine cellular immune responses to INO-3106 with and without INO-9012. Exploratory objectives included preliminary clinical efficacy to the therapy.

Four patients were consented and three patients met all inclusion and exclusion criteria and were enrolled to the study. Study therapy was well-tolerated, with no related serious adverse events and all related adverse events (AEs) being low-grade. Injection site pain was the most common related AE reported in all patients. Immunogenicity was evidenced by multiple immune assays shows engagement and expansion of an HPV6-specific cellular response, including hallmarks of cytotoxic T cells. Preliminary efficacy was demonstrated in these patients in the form of change of surgery frequency for growth resection. Prior to intervention both patients required surgery approximately every 180 days. One patient demonstrated a greater than 3 fold increase in surgery avoidance (584 days) and another patient remains completely surgery free as of the last contact at 915 days, a greater than 5 fold increase in surgery interval.

The experiments presented herein show that INO-3106 with and without INO-9012 was well tolerated, immunogenic and demonstrated preliminary efficacy in patients with HPV6-associated RRP aerodigestive lesions.

The materials and methods used in these experiments are now described.

### Study Population

This was a prospective, open-label, phase 1 study. Male and female patients at least 18 years old were considered for enrollment. To be eligible, patients must have histologically documented HPV6-associated aerodigestive papilloma, premalignant lesion or have aerodigestive invasive malignancy with most recent therapy (e.g., radiation, chemotherapy) completed at least two months prior to first dose of study treatment. Patients must have ECOG 0-1, with liver, renal, hepatic and bone marrow function within normal range. Patients were excluded if there was evidence of immunosuppression or anticipated use of immunosuppressive agents, required use of systemic steroids, presence of cardiac pre-excitation syndromes, or were pregnant or breast-feeding. Written informed consent was obtained from each patient prior to performing any assessments. The clinical trial was conducted according to the ethical guidelines of the Declaration of Helsinki.

### Immunotherapy and Electroporation Using CELLECTRA® Device

INO-3106 is a DNA plasmid encoding for the E6 and E7 proteins of HPV type 6, formulated in sterile water for injection. INO-3106 comprises the nucleotide sequence of SEQ ID NO:1 encoding the amino acid sequence of SEQ ID NO:2. INO-9012 consists of a DNA plasmid encoding for synthetic human IL-12 (p35 and p40 subunits) also formulated in sterile water for injection. INO-9012 comprises the nucleotide sequence of SEQ ID NO:5 encoding the amino acid sequence of SEQ ID NO:6 (p35 subunit of IL-12); and the nucleotide sequence of SEQ ID NO:7 encoding the amino acid sequence of SEQ ID NO:8 (p40 subunit of IL-12). Both INO-3106 and INO-9012 were designed using proprietary technology (Inovio Pharmaceuticals, Inc.) as described previously (Yan et al., Vaccine. 2008; 26(40):5210-5; Yan et al., Vaccine. 2009; 27(3):431-40). The CELLECTRA® 2000 adaptive constant current electroporation device (Inovio Pharmaceuticals, Inc.) delivers three 52 ms controlled electric pulses, spaced in 1 s intervals, through a sterile, disposable array to the injection site. When inserted into tissue, the needle array centers around the site of immunotherapy injection and creates transient pores within the cell membrane to enhance cell transfection. INO-3106 with or without INO-9012 was delivered intramuscularly in a 1 mL volume followed immediately by EP with the CELLECTRA® device. Treatment or dose is defined as injection of DNA plasmids followed by EP.

### Study Design

Following informed consent, each patient was assigned a unique patient identification code. Screening procedures to determine eligibility and collect baseline characteristics were completed within 28 days prior to first dose. Patients received escalating doses of INO-3106, of which the first dose (Day 0) delivered 3 mg of INO-3106, the second dose (Week 3) delivered 6 mg of INO-3106, and the third (Week 6) and fourth (Week 9) doses delivered 6 mg of INO-3106 with 1 mg of INO-9012. Each dose was delivered three weeks apart to allow for observation of development of any grade 2 or higher related systemic adverse events (AEs). In total, participation for all patients included a 9-week treatment period followed by a 6 month long term follow-up period from the last dose.

The primary objective of the study was to evaluate the safety and tolerability of INO-3106 with and without INO-9012. The secondary objective was to determine the humoral and cellular immune responses to INO-3106 with and without INO-9012, and the exploratory objective was to assess preliminary clinical efficacy to the treatment, as well as to associate efficacy with immune cell infiltration in post-dose tissue, if possible.

The study was registered on ClinicalTrials.gov with the identifier NCT02241369. The study protocol conforms to the ethical guidelines of the 1975 Declaration of Helsinki and was reviewed and approved by the center's Institutional Review Board.

### Safety Assessments

Local and systemic adverse events (AEs), vital signs, 12-lead electrocardiograms (ECGs) and the development of laboratory abnormalities were monitored from the date of informed consent through the last follow-up visit. In particular, injection site reactions, including pain, itching, erythema, induration and bruising were assessed on the day of each treatment and for 7 consecutive days post-treatment. Patients were queried at each visit regarding the occurrence of new AEs or disease and use of concomitant medications. All events were graded in accordance with the Common Terminology Criteria for Adverse Events (CTCAE), version 4.03 and coded with MedDRA version 21.

Further enrollment and treatment were halted immediately if one third or more patients experienced a related event requiring expedited reporting; any patient experiences a serious adverse event (SAE), unexpected grade 4 toxicity, potentially life-threatening AE or death assessed as related to study treatment; three or more patients experience the same related grade 3 or 4 AE; or if any patient reports a grade 3 anaphylaxis.

Women of reproductive potential were required to complete a pregnancy test at screening and within 3 days prior to each dose. Treatment was discontinued in women upon any positive pregnancy test results. Laboratory parameters including hematology, coagulation, serum chemistry (including liver function) and creatine phosphokinase (CPK), were monitored throughout the study and assessed locally at the center.

### Antigenic 3D Modeling

Comparative models were constructed using Bioluminate (Release 2019-2, Schrödinger, New York, N.Y.) and visualized with Discovery Studio Visualizer (Dassault Systemes BIOVIA, San Diego, Calif.).

### Interferon Gamma ELISpot

Whole blood was collected in ACD-A tubes and peripheral blood mononuclear cells (PBMCs) were isolated within 24 hours of draw. Samples were collected at baseline, at the time of immunotherapy dosing, and at each follow up visit and PBMCs cryopreserved for immune analyses in batches. T cell and antibody responses to HPV6 E6 and E7 antigens were determined by interferon-γ ELISpot and ELISA, respectively, as described previously (Bagarazzi et al., Sci Transl Med. 2012; 4(155):155ra38).

### Flow Cytometry

PBMCs were recovered after cryopreservation overnight in cell culture medium and spun, washed and re-suspended the following day. After counting, 1×10⁶ PBMCs were plated into a 96-well plate in R10 medium from patients with sufficient sample. For antigen specific responses, cells were stimulated 5 days with a combination of peptides corresponding to HPV6 E6 and E7 that had been pooled at a concentration of 2 μg/ml, while an irrelevant peptide was used as a negative control (OVA) and concanavalin A was used as a positive control (Sigma-Aldrich). No co-stimulatory antibodies or cytokines were added to cell cultures at any point. At the end of the 5 day incubation period, cells were stained for CD3-BUV737, CD4-APC-Cy7, CD14-BUV395, CD-16-BUV395, CD137-APC, Granulysin-AF488 CD-19-BUV395, CD38-BV786, CD8-BV650, granzyme B-AF700 (BD Biosciences), Granzyme A-PECy7 (ThermoFisher), PD-1-PEDazzle, perforin-BV421, Ki67-BV605 and CD69-BV711 (BioLegend). Staining for extracellular markers (CD4, CD8, CD137, CD69, CD38, PD-1) occurred first, followed by permeabilization to stain for the remaining markers. CD3 was stained intracellularly to account for downregulation of the marker following cellular activation. Acquired data were analyzed using the FlowJo software version X.0.7 or later (Tree Star).

### Antigen-Specific PBMC Stimulation for Gene Expression Analysis

For short term stimulation—Cryopreserved PBMCs were thawed, rested overnight, and stimulated for 22 hours at 37° C., 5% CO₂ and 95% humidity with either DMSO (negative control) or HPV6 E6 and E7 overlapping peptide pools (OLPs). Following stimulation, culture supernatants were collected and stored at −20° C. Cells were then lysed using Buffer RLT (Qiagen) and stored at −80° C.

For long term stimulation—Cryopreserved PBMCs were thawed, rested overnight, and stimulated at 37° C., 5% CO₂ and 95% humidity for 11 days with HPV6 E6 and E7 OLPs. On days 1, 4, 6 and 8, fresh media containing IL-2 and IL-7 was added at 10 U/mL and 10 ng/mL, respectively. On day 11, PBMCs were washed and rested overnight at 37° C., 5% CO₂ and 95% humidity. Following overnight rest, PBMCs were re-stimulated with HPV6 E6 and E7 OLPs for 22 hours with either DMSO (negative control) or HPV6 E6 and E7 OLPs. At the end of the 22-hour stimulation, cell supernatants were collected and stored at −20° C. Cells were then lysed and stored at −80° C.

### Multiplexed Gene Expression Analysis

Cell lysates were thawed in batches of 12 as per manufacturer instructions and hybridized to capture probes and fluorophore-barcoded reporter probes using the nCounter (NanoString) GX Human Immunology V2 panel, which consists of 594 genes plus 15 internal reference controls. Samples were then placed in the automated nCounter Prep Station (Nanostring) for hybridization of capture probes to a translucent cartridge, after which gene expression was measured by the nCounter Digital Analyzer (Nanostring) via direct counts of reporter probes in each sample lane.

### Statistical Methods

Subjects who received at least one dose of treatment were included in the safety analyses. The incidence of AEs, including SAEs and injection site reactions, was estimated along with exact 95% confidence intervals. Analyses related to secondary and exploratory endpoints will utilize subjects who received their assigned number of doses. In secondary analyses, immune response parameters will be estimated. In exploratory analyses, clinical response and histopathological assessment parameters will be estimated. For continuous outcomes, the mean/median and 95% confidence interval will be calculated, and for binary outcomes, the proportion and exact 95% confidence interval will be calculated using Clopper-Pearson methodology.

The results of the experiments are now described.

### Patient Characteristics and Disposition

In total, four patients were consented and screened for eligibility. Three patients met all inclusion and exclusion criteria and were enrolled from October 2014 to September 2017. Demographics and baseline characteristics are summarized in Table 1. Of those three, two patients presented with HPV6-associated RRP (both with disease in the vocal cords) and one patient had an invasive malignancy (initial disease located in trachea with squamous cell carcinoma in the oropharynx noted at study entry). All three patients completed all four doses, receiving 3 mg of INO-3106 on Day 0, 6 mg of INO-3106 at Week 3, and 6 mg of INO-3106 with 1 mg of INO-9012 at Weeks 6 and 9, all delivered intramuscularly via the CELLECTRA® device. Two patients completed the 6 month long term follow-up period following their last dose of treatment. One patient did not complete the long term follow-up, reporting a non-study related conflict as the primary reason for withdrawal from the study. All three patients are included in the safety analysis set.

TABLE 1

| Baseline Characteristic | Total (n = 3) |
|---|---|
| Age (yrs) | |
| Mean (SD) | 61.6 (10.5) |
| Median | 69.0 |
| Min, Max | 41.0, 75.0 |
| Gender (n, %) | |
| Male | 2 (66.7) |
| Female | 1 (33.3) |
| Race (n, %) | |
| White | 2 (66.7) |
| Black or African American | 1 (33.3) |
| Ethnicity (n, %) | |
| Non-Hispanic or Latino | 3 (100) |
| Weight (kg) | |
| Mean (SD) | 79.7 (14.5) |
| Median | 80.7 |
| Min, Max | 54.2, 104.4 |
| Height (cm)* | |
| Mean (SD) | 177.8 (15.2) |
| Median | 177.8 |
| Min, Max | 162.6, 193.0 |
| Body Mass Index (kg/m²)* | |
| Mean (SD) | 29.2 (1.3) |
| Median | 29.2 |
| Min, Max | 28.0, 30.5 |

*Height and body mass index not available for one patient (total n = 2).

Safety and Tolerability of INO-3106 and INO-9012 with EP

INO-3106 and INO-9012 delivered via EP was well-tolerated. Treatment-emergent AEs included injection site pain (three related grade 1 events), pyrexia (one unrelated grade 1 event) and urinary tract infection (one unrelated grade 2 event). All patients reported injection site pain, in most cases treated with medication leading to resolution. One treatment-emergent SAE of grade 3 monoplegia requiring hospitalization was reported on the study but was assessed to be unrelated to study treatment. No patients withdrew from receiving continued study treatment or from continued participation in the study due to an AE nor due to intolerability of EP. No grade 4 events nor deaths were reported during the course of the study. All patients experienced changes in laboratory parameters, the majority of which included slight fluctuations in hematology values, but all abnormal laboratory values were determined to be not clinically significant.

INO-3106 Induces IFNγ Production as Well as the Expression of Activation Markers and Lytic Proteins in T Cells from Treated RRP Patients

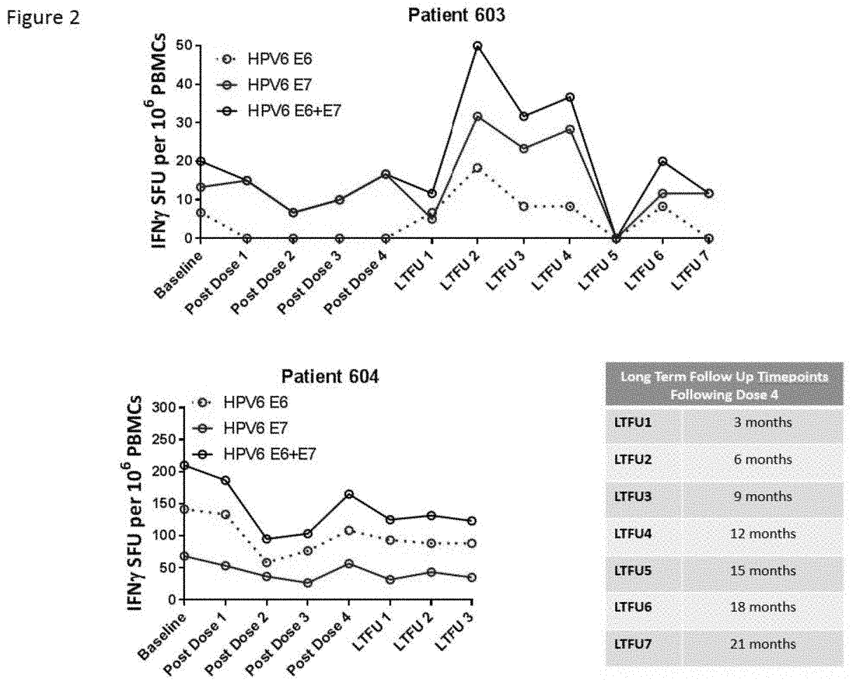

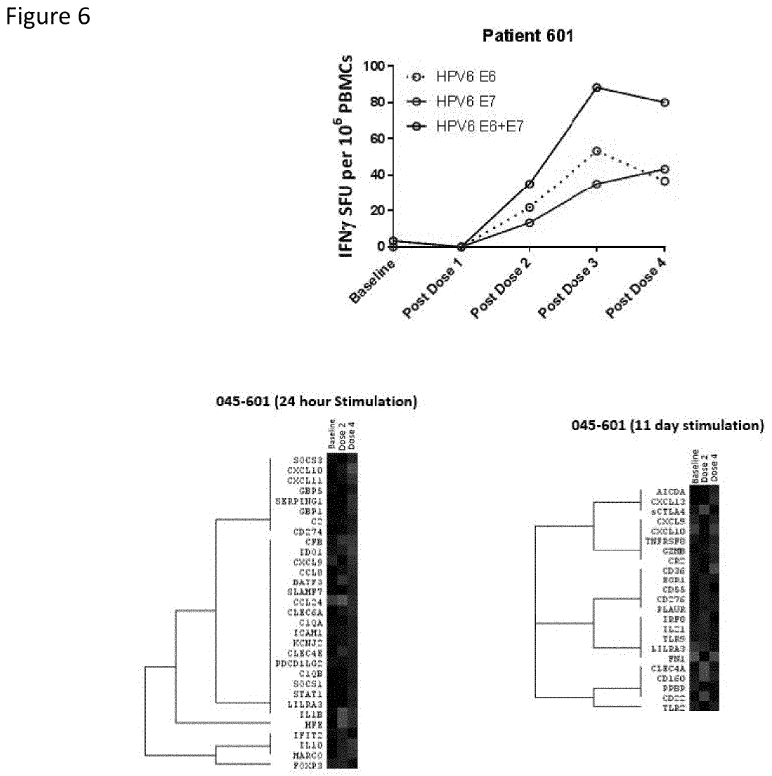

Assessment of HPV6 E6 and E7 cellular immune responses was performed for all three patients enrolled on the trial (FIG. 2 and FIG. 6). As subject 601 was not an RRP patient and has limited data due to death related to non-treatment events, immunology information related to this subject can be found in FIG. 6. Cellular immune responses were first addressed by performing an overnight IFNγ ELISpot without the addition of supportive cytokines on isolated peripheral blood mononuclear cells (PBMC) obtained prior to and following INO-3106 dosing. Results of this assessment suggest that patient 603 showed very low baseline activity to HPV6 E6 and E7 antigens in the form of antigen specific IFNγ secretion (FIG. 2). Specifically, less than 20 spots per 10⁶ PBMC were noted for the E6 or E7 antigen, whereas patient 604 showed reasonably robust cellular responses against these antigens at study entry, with E6 spot forming units per 10⁶ PBMC approaching 150 spots and E7 exceeding 50 spots (FIG. 2).

Treatment with INO-3106 increased HPV6 E6 and E7 specific cellular responses above baseline in patient 603, including total response to HPV6 antigens exceeding 50 spots. Patient 604 did not show an elevation in IFNγ spots in response to treatment but (FIG. 2). Of the responding patients, time to peak response varied and was difficult to accurately assess. Specifically, death occurred in patient 601 due to non-treatment related events after the fourth dose of INO-3106, thus no post-treatment follow-up was available and peak response was noted after the third dose. Patient 603 showed a peak response 6 months following their final dose of INO-3106, which may be related to changes in viral activation/antigenic target expression during that time or may reflect the kinetics of the patient's immune system to continue to build and support a large pool of HPV6 specific T cells.

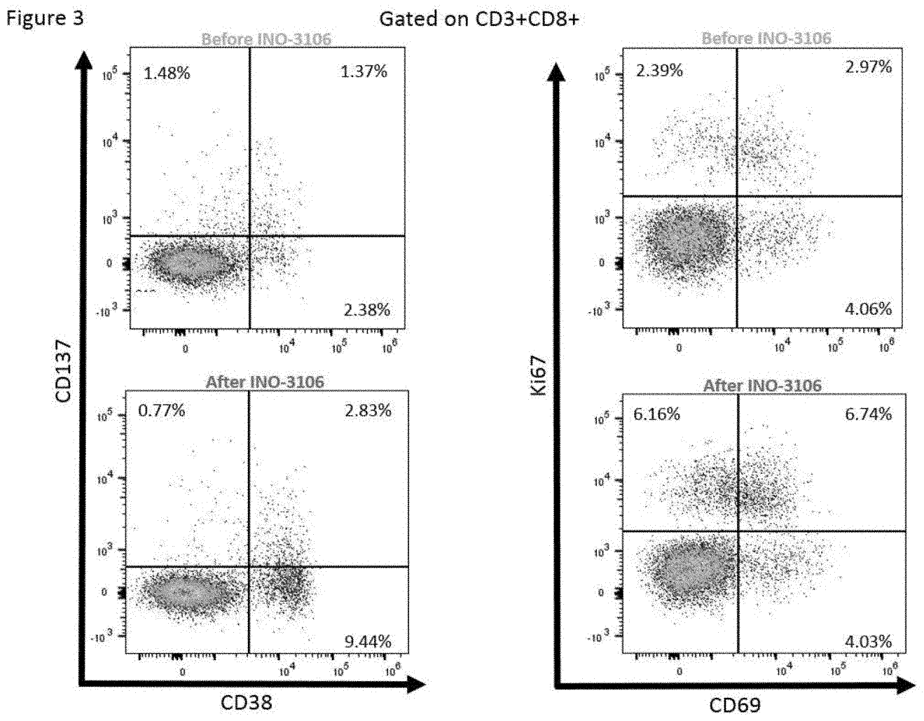

It has been shown previously that the production of IFNγ is suggestive of a Th1 immune response but does not correlate 1:1 with lytic activity (Morrow et al., Molecular therapy: the journal of the American Society of Gene Therapy. 2015; 23(3):591-601, Morrow et al., Clinical cancer research: an official journal of the American Association for Cancer Research. 2017; DOI: 10.1158/1078-0432.CCR-17-2335; Trimble et al., Lancet. 2015; 386(10008):2078-88; Migueles et al., PLoS pathogens. 2011; 7(2):e1002002; Varadarajan et al., The Journal of clinical investigation. 2011; 121(11):4322-31). It is understood that a cytolytic response by CD8+ T cells is a key component of an immune response that will control and eliminate virally infected cells. Therefore, flow cytometry was performed on PBMCs from patients 603 and 604 with sufficient sample isolated prior to and after dosing with INO-3106 to assess the ability of HPV6 specific CD8+ T cells to load granzymes and perforin in response to treatment. To that end, the CD8+ T cell compartment was analyzed for immune activation via antigen-specific expression of cell surface markers such as CD38, CD69, CD137 and Ki67 (FIG. 3) as well as for lytic potential as determined by the presence of granulysin (Gnly), granzyme A (GrzA), granzyme B (GrzB) and perforin (Prf) after in vitro stimulation with cognate antigens. Table 2 shows antigen specific regulation of these markers prior to and following treatment with INO-3106. Consistent with ELISpot responses, patient 603 exhibited robust elevations of a variety of CD8+ T cells expressing activation markers concomitant with lytic proteins. Most notably, expression of CD38 and/or Ki67 in combination with markers of lytic potential such as granzyme A, granzyme B and perforin increase dramatically after treatment with INO-3106, reaching values surpassing 3% of total CD8+ T cells being specific to HPV6 E6 and E7 antigens (Table 2A, FIG. 3). Conversely, and consistent with the ELISpot results suggesting the lack of a robust T cell expansion, patient 604 (Table 2B, FIG. 3) showed smaller elevations in CD8+ T cell responses from a magnitude perspective when compared to patient 603. Interestingly, while smaller in magnitude than patient 603, the phenotypes of the putative CTLs induced in patient 604 suggest the possibility of a more highly active CD8+ T cell, as the populations that were most likely to be increased after treatment consisted of three (CD38, CD137, Ki67) activation markers or concomitant expression of all four (CD69 in addition to the previous three). Co-expression of this number of activation markers concomitantly is far more rare (Tebas et al., The New England journal of medicine. 2017; Bagarazzi et al., Sci Transl Med. 2012; 4(155):155ra38; Morrow et al., Molecular therapy oncolytics. 2016; 3(16025; Trimble et al., Lancet. 2015; 386(10008):2078-88; vMorrow et al., Clinical cancer research: an official journal of the American Association for Cancer Research. 2018; 24(2):276-94; Aggarwal et al., Clinical cancer research: an official journal of the American Association for Cancer Research. 2019; 25(1):110-24) and previous interrogation of CD8+ T cells expressing multiple activation markers has suggested these cells expresses granzymes and can effectively induce apoptosis in targets expressing cognate antigen (Duhen et al., Nat Commun. 2018 Jul. 13; 9(1):2724. doi: 10.1038/s41467-018-05072-0). Indeed, this last idea is further supported when one notes that patient 604 showed increases in expression not just in granzymes and perforin, but in granulysin as well. While the limitations of this small sample size need to be taken into context, the results of this assessment suggests that INO-3106 potentially led to the induction of CD8+ T cells capable of activation in the context of antigenic exposure and that these cells were capable of granzyme, perforin and granulysin synthesis, thus exhibiting a clear HPV6 specific CTL phenotype.

TABLE 2A
Patient 603

| | | | Increase from |
|---|---|---|---|
| Markers co-expressed on | Before | After | Before to After |
| CD8+ T cells: | INO-3106 (%) | INO-3106 (%) | (% of CD8) |
| CD38 + Ki67+ | 0.49 | 3.95 | 3.46 |
| Ki67+ | 0.45 | 3.88 | 3.44 |
| CD38 + Ki67 + Prf+ | 0.53 | 3.93 | 3.40 |
| CD38 + Ki67 + GrzA + Prf+ | 0.58 | 3.95 | 3.37 |
| Ki67 + GrzA + Prf+ | 0.53 | 3.91 | 3.37 |
| Ki67 + Prf+ | 0.48 | 3.84 | 3.36 |
| Ki67 + GrzB + Prf+ | 0.50 | 3.66 | 3.16 |
| CD38 + Ki67 + GrzA + GrzB + Prf+ | 0.51 | 3.66 | 3.15 |
| CD38 + Ki67 + GrzB + Prf+ | 0.51 | 3.66 | 3.15 |
| Ki67 + GrzA + GrzB + Prf+ | 0.51 | 3.66 | 3.15 |

Key:
Prf = perform,
GrzA = Granzyme A,
GrzB = Granzyme B,
Gnly = Granulysin

TABLE 2B
Patient 604

| | | | Increase from |
|---|---|---|---|
| Markers co-expressed on | Before | After | Before to After |
| CD8 + T cells: | INO-3106 (%) | INO-3106 (%) | (% of CD8) |
| CD38 + CD137 + Ki67 + GrzB + Prf+ | 0.35 | 0.51 | 0.16 |
| CD38 + CD69 + CD137 + | 0.35 | 0.48 | 0.13 |
| Ki67 + GrzB + Prf+ | | | |
| CD137 + Ki67 + GrzB + Prf+ | 0.36 | 0.48 | 0.13 |
| CD38 + CD137 + Ki67 + | 0.36 | 0.48 | 0.12 |
| GrzA + GrzB + Prf+ | | | |
| CD69 + CD137 + Ki67 + GrzB + Prf+ | 0.36 | 0.47 | 0.11 |
| CD38 + CD69 + CD137 + Ki67 + | 0.36 | 0.46 | 0.09 |
| GrzA + GrzB + Prf+ | | | |
| CD137 + Ki67 + GrzA + GrzB + Prf+ | 0.37 | 0.46 | 0.09 |
| CD38 + CD137 + GrzB + Prf+ | 0.41 | 0.49 | 0.08 |
| CD38 + Ki67 + Gnly + GrzA + Prf+ | 0.00 | 0.08 | 0.08 |
| CD38 + Gnly+ | 0.00 | 0.08 | 0.08 |

Key:
Prf = perform,
GrzA = Granzyme A,
GrzB = Granzyme B,
Gnly = Granulysin

### INO-3106 Changes Immune Transcriptional Profiles of T Cells in RRP Patients

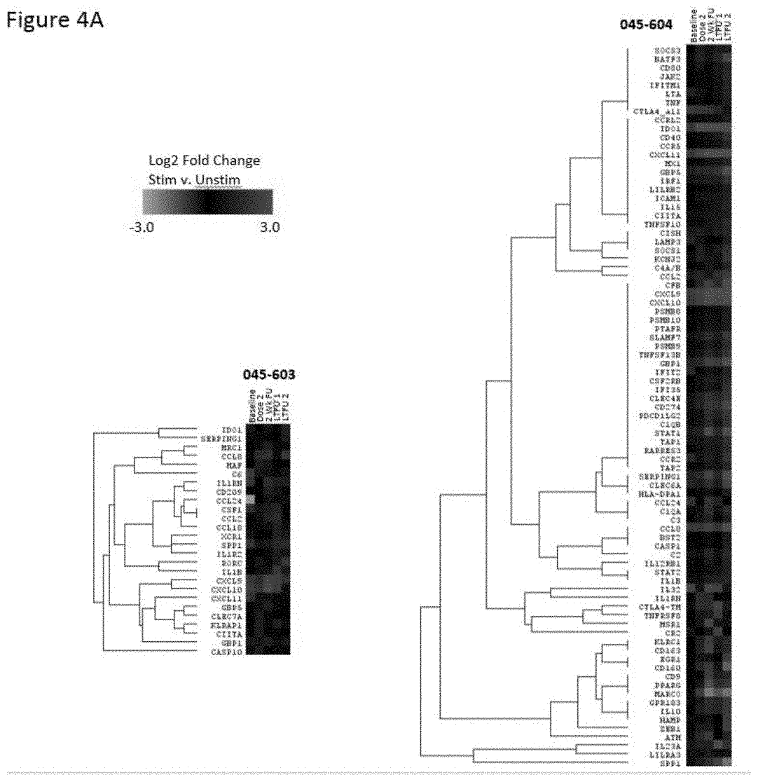

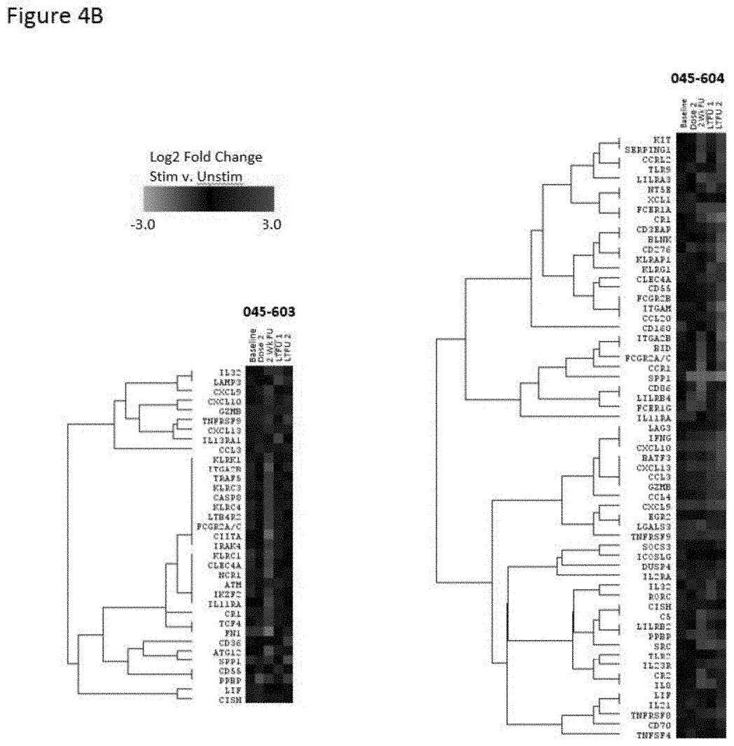

Short-term stimulations of patient PBMCs (24 hours) was performed, followed by an analysis of immune gene transcripts that were found to be specifically regulated in response to stimulation with HPV6 E6 and E7-derived peptide pools. Data from patient 601 can be found in FIG. 6. For patients 603 and 604, gene transcription was mostly associated with upregulation of a pro-inflammatory signature post immunotherapy. Patient 603 exhibited only modest differential gene expression at dose 2 compared to baseline. However, at the 2-week follow-up visit, a pronounced upregulation of genes associated with innate immune responses (CXCL10, CXCL9, CCL7, CCL8), genes associated with the IFNγ pathway (GBP1, GBP5), cell-cell interaction (CD209, MRC1) and B cell help (CXCL13) was observed in cells stimulated with peptide pool compared to unstimulated cells. Patient 604 also exhibited gene upregulation at the 2-week follow-up visit, with a similar signature as was observed in patient 603 (CXCL10, CXCL9, Stat1, GBP1, GBP5, CCL8). In addition, for patient 604, upregulation of markers indicative of adaptive cell activation (CD274, TNFSF13B) was observed. While gene upregulation in patient 603 was transient, patient 604 largely maintained this signature at the late follow up visits, three and six months post dose 4 (FIG. 4A, Table 3). Moreover, notably for patient 604, expression of IDO1, a molecule expressed by antigen presenting cells, increased rapidly over time, exhibiting a ˜4-fold difference at baseline that increased to 8-fold at dose 2, 10-fold at 2 week follow-up, almost 13-fold at 3 months follow up, and peaking at 65-fold increase at 6 months follow up (FIG. 4A, Table 3).

In vitro culture for 11 days followed by 24 h antigen-restimulation allowed for a look at the antigen-specific T cells. The stimulation conditions favor T cell expansion above all other cell types including B cells and other APCs. Analysis revealed reduced levels of differential gene expression compared to after ex vivo stimulation. Overall, both RRP patients showed similar patterns of gene upregulation with few genes upregulated at baseline (patient 603-0 genes and patient 604-7 genes), but increased differential expression at post-immunotherapy time points (patient 603-4 genes at dose 4, 7 genes at 2-week follow-up, 3 genes at 3 and 6 months follow-up, respectively; patient 604-12 genes at dose 2, 9 genes at 2-week follow-up, 10 genes at 3 months and 22 genes 6 months follow-up) (FIG. 4B). For both RRP patients, upregulated gene expression profiles in stimulated cells from post immunotherapy samples were primarily associated with T cell activation and functionality (CD276, TNFRSF8, TNFRSF9, GZMB) as well as B cell help (IL-21, CXCL13). See Table 4 for a full list of differentially expressed genes for each subject enrolled in the trial. Increased expression of CXCL10 and CXCL9 was also observed post immunotherapy for all subjects, however in patient 604 these markers were already increased at baseline.

### INO-3106 Reduces the Need for Surgical Intervention for the Treatment of RRP

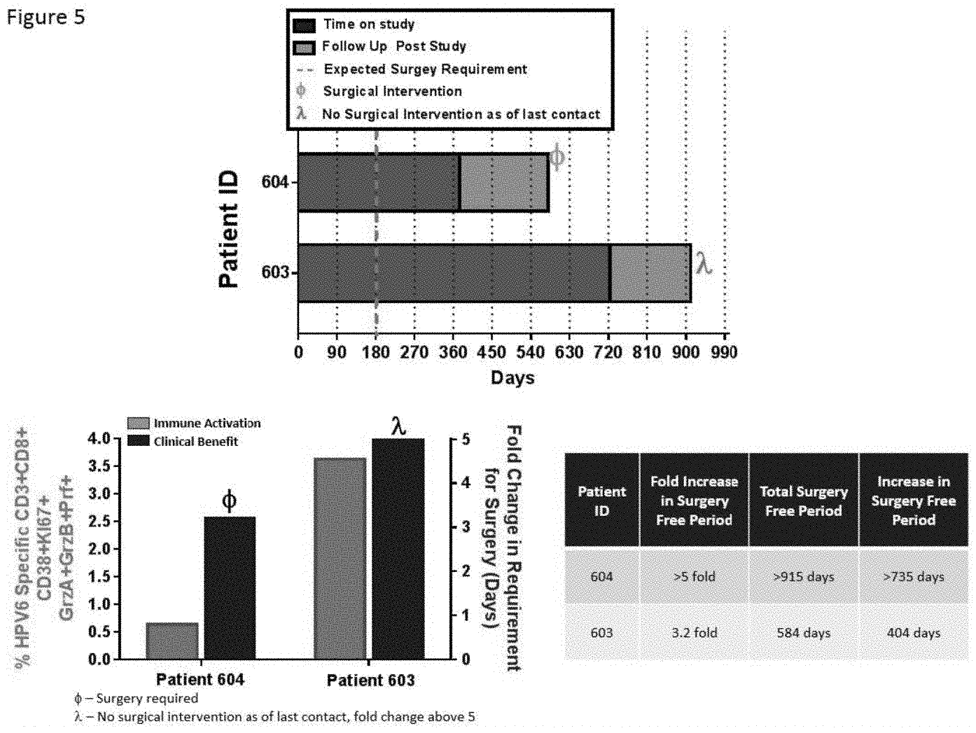

Prior to entry into the study, subjects 603 and 604 required surgical intervention to remove respiratory papillomas approximately every 180 days. Assuming this pattern were to continue, the expected number of required surgical interventions over the course of the study would be four for subject 603 and two for subject 604. However, over the entirety of the study neither subject required surgical intervention for the removal of airway papillomas, constituting a clinical change in the need for intervention in the treatment of this disease. Post-study follow up of these subjects reveals that subject 603 has not required surgical intervention in the treatment of disease at the time of this publication, totaling more than 915 days without surgery. After 584 days subject 604 had a recurrence of disease that did require surgical intervention to appropriately treat, an overall reduction in surgery frequency great than 3-fold (FIG. 5). The difference in the outcomes of these patients prompted examination if any of the immunology data generated while on study would have suggested a differential clinical response to treatment. Assessment of flow cytometry indicated that subject 604 had more robust immune activity in the form of HPV6-specific CTLs than subject 603 (FIG. 5). While not wishing to be bound by any particular theory, it is therefore possible that the difference in both the magnitude of response and pattern of activation marker expression on subject CTLs could associated with the durability of clinical impact.

Reported herein are the results of a phase I safety and immunology clinical trial of a HPV6 E6/E7 specific targeted DNA immunotherapy with and without IL-12 DNA adjuvant administered intramuscularly and delivered via electroporation by the CELLECTRA® device in three patients with HPV6 associated aerodigestive precancerous lesions and malignancies. Administration of the immunotherapy was well-tolerated. There were no treatment-related SAEs and the most frequent treatment-emergent AEs were injection site reactions. All patients showed induction of cellular responses to the HPV6 E6 and E7 antigens as demonstrated by at least one immunological assessment. Notably, both evaluable RRP patients derived clinical benefit from treatment with INO-3106, mainly in the form of delayed treatment intervention (e.g., surgery) relative to their pre-study surgery frequencies. Moreover, the fact that the patient whom exhibited more robust cellular activity after INO-3106 treatment remains surgery free while the patient with less robust cellular activity delayed but did not completely avoid surgery suggests a possible causal relationship between the induction of an HPV6-specific cellular response and the type/duration of clinical benefit. These results are encouraging and present the idea that, in certain instances, additional dosing to continue to boost the cellular response may be preferable in this treatment setting. Treatment with INO-3106 resulted in the induction of HPV6-specific cellular responses across a variety of immunoassays. The confirmation of production of IFNγ using ELISpot as well as the confirmation of expression of activation markers concomitant with synthesis of granzyme and perforin on CD8+ T cells via flow cytometry suggests that INO-3106 drove the induction of a proinflammatory immune response that included T cells with hallmarks of highly activated cytotoxic lymphocytes. These results are further underscored by the observation of dynamic regulation of pro-inflammatory as well as regulatory gene transcripts in PBMCs after completion of treatment. Specifically, genes associated with the IFNγ pathway such as CXCL10 and GBP1 were upregulated after both short and long-term stimulation. A recent study in squamous cell carcinoma of the head and neck reported that a composite score based on IFNγ, CXCL9, CXCL10, IDO1 HLA-DRA and STAT1 significantly correlated with treatment response rate, indicating that an IFNγ-based signature is associated with treatment benefit (Ahn et al., Laryngoscope. 2018; 128(1):E27-E32). Moreover, increased expression of Granzyme B and TNFRSF9 was observed, confirming the activity of cytotoxic lymphocytes on the transcriptomic level. The necessity for a T cell response of this nature in combating HPV-driven disease has been exemplified in two previous clinical trials for DNA-based immunotherapy, both of which were delivered using the CELLECTRA® device. In the context of HPV-associated cervical dysplasia, clinical response to treatment with VGX-3100 (DNA immunotherapy for HPV16/18) in the form of regression of lesions concomitant with elimination of HPV infection was statistically associated with the presence of a robust cellular response that included IFNγ and CD8+ T cells exhibiting phenotypic markers of cytoxicity (Trimble et al., Lancet. 2015; 386(10008):2078-88). Additionally, in another trial investigating treatment of HPV-associated squamous cell cancer of the oropharynx, a patient with metastatic cancer who achieved a complete response to treatment with nivolumab after treatment with MEDI0457 (DNA immunotherapy for HPV16/18 with plasmid encoded IL-12 adjuvant) was noted as having a therapy-driven robust expansion of PD1+ cytotoxic T cells (Aggarwal et al., Clinical cancer research: an official journal of the American Association for Cancer Research. 2019; 25(1):110-24). Thus, the current study provides further evidence that HPV-specific immunotherapies delivered by the CELLECTRA® device induce the generation of potent T cell responses that have the potential to clinically impact HPV-associated tumorigenesis.

The data collected from this study are the first to suggest that an HPV specific immunotherapy may be able to impact the clinical status of patients with HPV6 associated recurrent respiratory papillomatosis and act as an additional or alternative adjuvant therapy. These findings are complementary to data presented earlier this year, where administration of pembrolizumab was associated with a reduced need for routine surgical interventions (Pai et al., Journal of Clinical Oncology. 37. 2502-2502. 10.1200/JCO.2019.37.15_supp1.2502). Together these findings offer early data to support the use of immunotherapeutic approaches in the management of these patients. The standard of care for treatment for this disease is repeated surgical intervention, which presents a number of complications and is unlikely to completely eradicate lesion recurrence as latent virus may reside in adjacent tissue (Chow et al., APMIS. 2010; 118(6-7):422-49). Other non-surgical adjuvant interventions are indicated in patients with rapid regrowth of lesions or aggressive disease, but such therapies also carry inherent risks and require further evaluation to determine optimal treatment regimens (Derkay et al., Otolaryngol Clin North Am. 2019; 52(4):669-79). Current treatment limitations highlight the need to identify non-invasive and immune mediated approaches to treating patients with HPV positive areodigestive disease. Indeed, preventive HPV vaccines have been reported to reduce papilloma growth and extend time between interventions, however, determination of therapeutic efficacy requires continued evaluation (Makiyama et al., J Voice. 2017; 31(1):104-6). Similarly, PD-1/PD-L1 inhibition represents a rational approach to treating RRP, but expression and impact on clinical outcome is less characterized (Ahn et al., Laryngoscope. 2018; 128(1):E27-E32).

The data generated from the present study suggests that immunotherapy with INO-3106 and IL-12 adjuvant as a non-invasive immune mediated approach may provide an option to address existing treatment deficiencies for RRP.

1. A method for treating or preventing recurrent respiratory papillomatosis (RRP) in an individual comprising administering to the individual a composition comprising a nucleic acid molecule encoding an HPV6 antigen.

2. The method of claim 1, wherein the HPV6 antigen is an HPV6 E6-E7 fusion antigen.

3. The method of claim 2, wherein the nucleic acid molecule comprises one or more nucleotide sequences selected from the group consisting of:
a nucleotide sequence that encodes SEQ ID NO:2;
a nucleotide sequence that is at least 95% homologous to a nucleotide sequence that encodes SEQ ID NO:2;
a fragment of a nucleotide sequence that encodes SEQ ID NO:2;
a nucleotide sequence that is at least 95% homologous to a fragment of a nucleotide sequence that encodes SEQ ID NO:2.

4. The method of claim 3, wherein the nucleic acid molecule comprises a nucleotide sequence that is at least 98% homologous to a nucleotide sequence that encodes SEQ ID NO:2.

5. The method of claim 3, wherein the nucleic acid molecule comprises a nucleotide sequence that is at least 99% homologous to a nucleotide sequence that encodes SEQ ID NO:2.

6. The method of claim 3, where the nucleotide sequences encoding the HPV6 E6-E7 fusion antigen are without a leader sequence at 5′ end that is a nucleotide sequence that encodes SEQ ID NO:4.

7. The method of claim 3, wherein the nucleic acid molecule comprises one or more nucleotide sequences selected from the group consisting of:
a nucleotide sequence comprising SEQ ID NO:1;
a nucleotide sequence that is at least 95% homologous SEQ ID NO:1;
a fragment of SEQ ID NO:1;
a nucleotide sequence that is at least 95% homologous to a fragment of SEQ ID NO:1.

8. The method of claim 3, wherein the nucleic acid molecule comprises a nucleotide sequence that is at least 98% homologous to SEQ ID NO:1.

9. The method of claim 3, wherein the nucleic acid molecule comprises a nucleotide sequence that is at least 99% homologous to SEQ ID NO:1.

10. The method of claim 3, wherein said nucleic acid molecule is a plasmid.

11. The method of claim 3, wherein the composition is a pharmaceutical composition.

12. The method of claim 1, further comprising administering to the individual a composition comprising an adjuvant.

13. The method of claim 1, further comprising administering to the individual a nucleic acid molecule comprising a nucleotide sequence encoding one or more of: p35 and p40 subunits of IL-12.

14. The method of claim 13, wherein the nucleotide sequence encoding p35 comprises a nucleotide sequence selected from the group consisting of:
a nucleotide sequence that encodes SEQ ID NO:6;
a nucleotide sequence that is at least 95% homologous to a nucleotide sequence that encodes SEQ ID NO:6;
a fragment of a nucleotide sequence that encodes SEQ ID NO:6;
a nucleotide sequence that is at least 95% homologous to a fragment of a nucleotide sequence that encodes SEQ ID NO:6.

15. The method of claim 13, wherein the nucleotide sequence encoding p40 comprises a nucleotide sequence selected from the group consisting of:
a nucleotide sequence that encodes SEQ ID NO:8;
a nucleotide sequence that is at least 95% homologous to a nucleotide sequence that encodes SEQ ID NO:8;
a fragment of a nucleotide sequence that encodes SEQ ID NO:8;

16. a nucleotide sequence that is at least 95% homologous to a fragment of a nucleotide sequence that encodes SEQ ID NO:8.

17. The method of claim 3, wherein administering said nucleic acid molecule to the individual comprises electroporation.


SEQUENCE LISTING (8 sequences)

SEQ ID NO: 1 - DNA, 873 bp, Artificial Sequence
  HPV6 E6E7 DNA sequence
ggtaccggatccgccaccatggactggacctggattctgttcctggtggccgctgccacacgggtgcacagcgaaagcgccaatgccagcaccagcgctacaaccatcgaccagctgtgcaagaccttcaacctgagcatgcacaccctgcagatcaactgcgtgttctgcaagaacgccctgaccaccgccgagatctacagctacgcctacaagcagctgaaggtgctgttcagaggcggctacccctatgccgcctgcgcctgctgcctggaatttcacggcaagatcaaccagtaccggcacttcgactacgccggctacgccaccaccgtggaagaggaaacaaagcaggacatcctggacgtgctgatccggtgctacctgtgccacaagcccctgtgcgaggtggaaaaagtgaagcacatcctgaccaaggcccggttcatcaagctgaactgcacctggaagggccggtgcctgcactgctggaccacctgtatggaagatatgctgcccagaggccggaagcggcggagccatggcagacacgtgaccctgaaggacatcgtgctggacctgcagccccccgatcctgtgggcctgcactgttacgagcagctggtggacagcagcgaggacgaggtggacgaagtggacggccaggacagccagcccctgaagcagcacttccagatcgtgacctgctgctgcggctgcgacagcaacgtgcggctggtggtgcagtgcaccgagacagacatcagagaggtccagcagctcctgctgggcaccctgaacatcgtgtgccccatctgcgcccccaagacctacccttacgacgtgcccgactacgcctgatgactcgagctc

SEQ ID NO: 2 - protein, 280 aa, Artificial Sequence
  HPV6 E6E7 protein sequence
MDWTWILFLVAAATRVHSESANASTSATTIDQLCKTFNLSMHTLQINCVFCKNALTTAEIYSYAYKQLKVLFRGGYPYAACACCLEFHGKINQYRHFDYAGYATTVEEETKQDILDVLIRCYLCHKPLCEVEKVKHILTKARFIKLNCTWKGRCLHCWTTCMEDMLPRGRKRRSHGRHVTLKDIVLDLQPPDPVGLHCYEQLVDSSEDEVDEVDGQDSQPLKQHFQIVTCCCGCDSNVRLVVQCTETDIREVQQLLLGTLNIVCPICAPKTYPYDVPDYA

SEQ ID NO: 3 - DNA, 54 bp, Artificial Sequence
  IgE Leader DNA sequence
atggactggacctggatcctgttcctggtggccgctgccacacgggtgcacagc

SEQ ID NO: 4 - protein, 18 aa, Artificial Sequence
  IgE leader protein
MDWTWILFLVAAATRVHS

SEQ ID NO: 5 - DNA, 660 bp, Artificial Sequence
  p35 DNA sequence
atgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 6 - protein, 219 aa, Artificial Sequence
  p35 amino acid sequence
MCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 7 - DNA, 987 bp, Artificial Sequence
  p40 DNA sequence
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttag

SEQ ID NO: 8 - protein, 328 aa, Artificial Sequence
  p40 amino acid sequence
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS